SEQUENCE LISTING (3 sequences)

SEQ ID NO: 1 - DNA, 21 bp, synthetic construct
  source 1..21: mol_type = other DNA
aatctcgtggccttaatgaaa

SEQ ID NO: 2 - DNA, 21 bp, synthetic construct
  source 1..21: mol_type = other DNA
aatctcgtggccttaatgaag

SEQ ID NO: 3 - RNA, 21 nt, synthetic construct
  source 1..21: mol_type = other RNA
tttcattaaggccacgagatt

(19) United States
(12) Patent Application Publication
(10) Pub. No.: US 2025/0250571 A1
(43) Pub. Date: Aug. 7, 2025

(54) DOUBLE-STRANDED RNA MOLECULE FOR ADMINISTRATION TO THE EYE

(71) Applicant: Hoffmann-La Roche Inc., Little Falls, NJ (US)

(72) Inventors: Konrad BLEICHER, Freiburg (DE); Erik FUNDER, Hillerød (DK); Helle JACOBSEN, Virum (DK); Dennis Jul HANSEN, Farum (DK); Michael KELLER, Basel (CH); Inna Appeldorff LARSEN, Kokkedal (DK); Tiago Francisco SANTOS FERREIRA, Basel (CH); Lotte WINTHER, Vejle (DK); Jesper WORM, Copenhagen (DK); Lena WYSS, Basel (CH)

(21) Appl. No.: 19/056,125

(22) Filed: Feb. 18, 2025

Related U.S. Application Data
(63) Continuation of application No. PCT/EP2023/074440, filed on Sep. 6, 2023.

(30) Foreign Application Priority Data
Sep. 6, 2022 (EP) 22194224.6

(51) Int. Cl. C12N 15/113; A61K 9/00; A61P 27/02

(52) U.S. Cl. CPC C12N 15/113; A61K 9/0048; A61P 27/02; C12N 2310/11; C12N 2310/14; C12N 2310/315; C12N 2310/321; C12N 2310/322; C12N 2310/3515

(57) ABSTRACT

The present invention relates to double-stranded RNA molecules conjugated to at least one conjugate moiety for topical administration to the eye, and pharmaceutical compositions thereof. The double-stranded RNA molecules are complementary, such as fully complementary, to targets expressed in the eye, and are capable of inhibiting expression of targets expressed in the eye. The double-stranded RNA molecules can be used in the treatment of conditions and diseases of the eye.

### SEQUENCE LISTING

The instant application contains a Sequence Listing which has been submitted electronically in XML format and is hereby incorporated by reference in its entirety. Said XML copy, created on Feb. 10, 2025, is named “51551-026001_Sequence_Listing_2_10_25.xml” and is 3,547 bytes in size.

### FIELD OF THE INVENTION

The present invention relates to double-stranded RNA molecules conjugated to at least one conjugate moiety for topical administration to the eye, and pharmaceutical compositions thereof; and their use in the treatment of conditions and diseases of the eye.

### BACKGROUND

Double-stranded RNA molecules such as siRNA molecules can modulate the expression of a target nucleic acid, in particular by inhibiting the expression of a target nucleic acid, by binding to complementary mRNA after transcription, typically leading to degradation and loss in translation of the target mRNA.

siRNA molecules are capable of inducing RNA-dependent gene silencing via the RNA-induced silencing complex (RISC) in a cell's cytoplasm, where they interact with the catalytic RISC component argonaute.

Delivery of double-stranded RNA molecules to tissue sites or specific cells can be impaired or prevented by a variety of factors which reduce the stability of double-stranded RNA molecules or prevent double-stranded RNA molecules from being effectively delivered to their target site. Such factors include, but are not limited to, susceptibility to endogenous RNases, short half-life and poor stability, recognition by the immune system, large size and charge and endosomal trapping.

One tissue site which can represent a particular challenge for delivery is the eye, which can be affected by a variety of conditions, pathologies and diseases. Delivery of double-stranded RNA molecules to the eye may result in beneficial therapeutic effects due to double-stranded RNA molecules targeting and inhibiting genes of interest expressed in the eye which may be involved in causing pathology.

There is a need for an effective mechanism for delivering double-stranded RNA molecules to the eye.

### SUMMARY OF INVENTION

The present invention provides a double-stranded RNA molecule for topical administration to the eye, wherein the double-stranded RNA molecule is capable of binding to a target sequence, wherein the double-stranded RNA molecule comprises a first strand having a 5′ end and a 3′ end, and a second strand having a 5′ end and a 3′ end, wherein the first strand is complementary to the second strand, wherein the first strand comprises a contiguous nucleotide sequence of at least 8 nucleotides in length which is complementary to a target sequence, and wherein the double-stranded RNA molecule is conjugated to at least one conjugate moiety.

In some embodiments, the double-stranded RNA molecule may be a small interfering RNA (siRNA) molecule. In some embodiments, the double-stranded RNA molecule may be a small hairpin RNA (shRNA) molecule.

In some embodiments, the double-stranded RNA molecule may be capable of inhibiting the expression of a target.

The double-stranded RNA molecule is conjugated to at least one conjugate moiety. In some embodiments the double-stranded RNA molecule may be conjugated to at least two or at least three conjugate moieties. In some embodiments the double-stranded RNA molecule may be conjugated to two conjugate moieties. In some embodiments the double-stranded RNA molecule may be conjugated to three conjugate moieties.

In some embodiments the conjugate moiety or conjugate moieties may be covalently attached to the double-stranded RNA molecule.

In some embodiments, the conjugate moiety may be a fatty acid molecule or a cholesterol molecule.

In embodiments where more than one conjugate moiety is present, the conjugate moieties may be a combination of one or more fatty acid molecule(s) and one or more cholesterol molecule(s), or may be a combination of two or more fatty acid molecules, or may be a combination of two or more cholesterol molecules. In embodiments where more than one conjugate moiety is present, each conjugate moiety is independently selected such that the conjugate moieties attached to the double-stranded RNA molecule may or may not be the same.

In some embodiments, the conjugate moiety may be a fatty acid molecule.

In some embodiments, the fatty acid molecule is selected from the list consisting of: C3, C4, C5, C6, C7, C8, C9, C10, C11, C12, C13, C14, C15, C16, C17, C18, C19, C20, C21, C22, C23, C24, C25, C26, C27, C28, C29, C30, C31, C32, C33, C34, C35, C36, C37, C38, C39 and C40.

In some embodiments, the fatty acid molecule may be C16.

In some embodiments, the fatty acid molecule may be C22.

In some embodiments, the fatty acid molecule is branched. In some embodiments, the fatty acid molecule is unbranched.

In some embodiments, the fatty acid molecule is saturated. In some embodiments, the fatty acid molecule is unsaturated.

In some embodiments, the fatty acid molecule comprises one or more double bonds. In some embodiments, the fatty acid molecule comprises two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more carbon double bonds.

In some embodiments, the fatty acid molecule comprises one or more triple bonds. In some embodiments, the fatty acid molecule comprises two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more carbon triple bonds.

In some embodiments, the fatty acid molecule is selected from the list consisting of: C3:0; C4:0; C4:1; C5:0; C5:1; C6:0; C6:1; C6:2; C7:0; C7:1; C7:2; C8:0; C8:1; C8:2; C8:3; C9:0; C9:1; C9:2; C9:3; C10:0; C10:1; C10:2; C10:3; C10:4; C11:0; C11:1; C11:2; C11:3; C11:4; C12:0; C12:1; C12:2; C12:3; C12:4; C12:5; C13:0; C13:1; C13:2; C13:3; C13:4; C13:5; C14:0; C14:1; C14:2; C14:3; C14:4; C14:5; C14:6; C15:0; C15:1; C15:2; C15:3; C15:4; C15:5; C15:6; C16:0; C16:1; C16:2; C16:3; C16:4; C16:5; C16:6; C16:7; C17:0; C17:1; C17:2; C17:3; C17:4; C17:5; C17:6; C17:7; C18:0; C18:1; C18:2; C18:3; C18:4; C18:5; C18:6; C18:7; C18:8; C19:0; C19:1; C19:2; C19:3; C19:4; C19:5; C19:6; C19:7; C19:8; C20:0; C20:1; C20:2; C20:3; C20:4; C20:5; C20:6; C20:7; C20:8; C20:9; C21:0; C21:1; C21:2; C21:3; C21:4; C21:5; C21:6; C21:7; C21:8; C21:9; C22:0; C22:1; C22:2; C22:3; C22:4; C22:5; C22:6; C22:7; C22:8; C22:9; C22:10; C23:0; C23:1; C23:2; C23:3; C23:4; C23:5; C23:6; C23:7; C23:8; C23:9; C23:10; C24:0; C24:1; C24:2; C24:3; C24:4; C24:5; C24:6; C24:7; C24:8; C24:9; C24:10; C24:11; C25:0; C25:1; C25:2; C25:3; C25:4; C25:5; C25:6; C25:7; C25:8; C25:9; C25:10; C25:11; C26:0; C26:1; C26:2; C26:3; C26:4; C26:5; C26:6; C26:7; C26:8; C26:9; C26:10; C26:11; C26:12; C27:0; C27:1; C27:2; C27:3; C27:4; C27:5; C27:6; C27:7; C27:8; C27:9; C27:10; C27:11; C27:12; C28:0; C28:1; C28:2; C28:3; C28:4; C28:5; C28:6; C28:7; C28:8; C28:9; C28:10; C28:11; C28:12; C28:13; C29:0; C29:1; C29:2; C29:3; C29:4; C29:5; C29:6; C29:7; C29:8; C29:9; C29:10; C29:11; C29:12; C29:13; C30:0; C30:1; C30:2; C30:3; C30:4; C30:5; C30:6; C30:7; C30:8; C30:9; C30:10; C30:11; C30:12; C30:13; C30:14; C31:0; C31:1; C31:2; C31:3; C31:4; C31:5; C31:6; C31:7; C31:8; C31:9; C31:10; C31:11; C31:12; C31:13; C31:14; C32:0; C32:1; C32:2; C32:3; C32:4; C32:5; C32:6; C32:7; C32:8; C32:9; C32:10; C32:11; C32:12; C32:13; C32:14; C32:15; C33:0; C33:1; C33:2; C33:3; C33:4; C33:5; C33:6; C33:7; C33:8; C33:9; C33:10; C33:11; C33:12; C33:13; C33:14; C33:15; C34:0; C34:1; C34:2; C34:3; C34:4; C34:5; C34:6; C34:7; C34:8; C34:9; C34:10; C34:11; C34:12; C34:13; C34:14; C34:15; C34:16; C35:0; C35:1; C35:2; C35:3; C35:4; C35:5; C35:6; C35:7; C35:8; C35:9; C35:10; C35:11; C35:12; C35:13; C35:14; C35:15; C35:16; C36:0; C36:1; C36:2; C36:3; C36:4; C36:5; C36:6; C36:7; C36:8; C36:9; C36:10; C36:11; C36:12; C36:13; C36:14; C36:15; C36:16; C36:17; C37:0; C37:1; C37:2; C37:3; C37:4; C37:5; C37:6; C37:7; C37:8; C37:9; C37:10; C37:11; C37:12; C37:13; C37:14; C37:15; C37:16; C37:17; C38:0; C38:1; C38:2; C38:3; C38:4; C38:5; C38:6; C38:7; C38:8; C38:9; C38:10; C38:11; C38:12; C38:13; C38:14; C38:15; C38:16; C38:17; C38:18; C39:0; C39:1; C39:2; C39:3; C39:4; C39:5; C39:6; C39:7; C39:8; C39:9; C39:10; C39:11; C39:12; C39:13; C39:14; C39:15; C39:16; C39:17; C39:18; C40:0; C40:1; C40:2; C40:3; C40:4; C40:5; C40:6; C40:7; C40:8; C40:9; C40:10; C40:11; C40:12; C40:13; C40:14; C40:15; C40:16; C40:17; C40:18; C40:19.

In embodiments where more than one fatty acid molecule is present, each fatty acid molecule is independently selected such that the fatty acid molecules attached to the double-stranded RNA molecule may or may not be the same.

In some embodiments, the conjugate moiety may be a cholesterol molecule.

In some embodiments, the cholesterol molecule may be selected from the group consisting of 3′-cholesteryl-TEG CPG, 5′-cholesterol-TEG-CE phosphoramidite, 5′-cholesterol-CE phosphoramidite and cholesteryl-TEG-CE phosphoramidite. In embodiments where more than one cholesterol moiety is present, the cholesterol moieties may be a combination selected from the group consisting of 3′-cholesteryl-TEG CPG, 5′-cholesterol-TEG-CE phosphoramidite, 5′-cholesterol-CE phosphoramidite and cholesteryl-TEG-CE phosphoramidite.

In embodiments where more than one cholesterol molecule is present, each cholesterol molecule is independently selected such that the cholesterol molecules attached to the double-stranded RNA molecule may or may not be the same.

In embodiments where more than one conjugate moiety is present, the conjugate moieties may be a combination of one or more selected from C16, C22 or a cholesterol molecule.

In some embodiments, the conjugate moiety may be positioned at the 5′ end or 3′ end of one of the strands of the double-stranded RNA molecule. In some embodiments, the conjugate moiety may be positioned at the 3′ end of the first strand of the double-stranded RNA molecule. The first strand may be in the sense orientation (i.e. the first strand is a sense strand). In some embodiments, the conjugate moiety may be positioned at the 3′ end of the sense strand of the double-stranded RNA molecule.

In some embodiments, a linker may be positioned between the double-stranded RNA molecule and the conjugate moiety. In some embodiments, the linker may be C6. In some embodiments, the linker may be TEG. In some embodiments, the linker is a dinucleotide. In some embodiments, the dinucleotide is CA (in other words, in some embodiments the linker is a CA dinucleotide).

In some embodiments wherein the double-stranded RNA molecule comprises more than one conjugate moiety, a linker may be positioned between the double-stranded RNA molecule and each of the conjugate moieties. In some embodiments a linker may be positioned between each of the conjugate moieties.

In some embodiments the linker may be a cleavable linker.

In some embodiments, the contiguous nucleotide sequence may be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

In some embodiments, the contiguous nucleotide sequence may be at least 20 nucleotides in length. In some embodiments, the contiguous nucleotide sequence may be 20, 21, 22, 23 or 24 nucleotides in length.

In some embodiments, the first strand may consist of the contiguous nucleotide sequence.

In some embodiments, the double-stranded RNA molecule may be for administration to the front of the eye.

In some embodiments, the double-stranded RNA molecule may be for administration to the conjunctiva of the eye or the cornea of the eye. In some embodiments, the double-stranded RNA molecule may be for administration to the bulbar conjunctiva, the palpebral conjunctiva, the ocular conjunctiva and/or the fornix conjunctiva.

In some embodiments, the contiguous nucleotide sequence may be at least about 75% complementary to a target sequence. The contiguous nucleotide sequence may be at least about 80%, at least about 85%, at least about 90%, at least about 95% or fully (such as about 100%) complementary to a target sequence. In some embodiments, the contiguous nucleotide sequence may comprise 1, 2, 3, 4, 5, 6, 7, 8 or more mismatches to a target sequence.

In some embodiments, the target may be AHA-1. In some embodiments, the AHA-1 target may comprise or consist of SEQ ID NO: 1 or SEQ ID NO: 2.

In some embodiments, the contiguous nucleotide sequence may be complementary to an AHA-1 target sequence. In some embodiments, the contiguous nucleotide sequence may comprise a nucleotide sequence that is complementary to SEQ ID NO: 1 or SEQ ID NO: 2. In some embodiments, the contiguous nucleotide sequence may comprise or consist of SEQ ID NO: 3.

In some embodiments, the expression of the target may be inhibited by about 10%, about 15%, about 20%, about 25%, about 30%, about 35%, about 40%, about 45%, about 50%, about 55%, about 60%, about 65%, about 70%, about 75%, about 80%, about 95% or about 100%, compared to a control. It will be understood that the % inhibition in the expression of a target referred to above will be a % decrease relative to a control, wherein the term “control” refers to expression of a target in a cell that has not been exposed to the double-stranded RNA molecule of the invention.

In some embodiments, the double-stranded RNA molecule may comprise one or more modified nucleoside(s). The one or more modified nucleoside may be one or more 2′ sugar modified nucleoside(s) independently selected from the group consisting of: 2′-O-alkyl-RNA; 2′-O-methyl RNA (2′-OMe); 2′-O-methoxyethyl-RNA (2′-MOE), 2′-alkoxy-RNA; 2′-amino-DNA; 2′-fluro-RNA; 2′-fluoro-DNA; arabino nucleic acid (ANA); 2′-fluoro-ANA; locked nucleic acid (LNA), and any combination thereof. In some embodiments, the 2′ sugar modified nucleoside may be an affinity enhancing 2′ sugar modified nucleoside.

In some embodiments, one or more of the internucleoside linkages positioned between the nucleosides on the contiguous nucleotide sequence may be modified. In some embodiments, at least about 75%, at least about 80%, at least about 85%, at least about 90%, at least about 95%, or about 100% of the internucleoside linkages positioned between the nucleosides on the contiguous nucleotide sequence may be modified.

In some embodiments, one or more, or all, of the modified internucleoside linkages may comprise a phosphorothioate linkage. In some embodiments, all the internucleoside linkages present in the double-stranded RNA molecule may be phosphorothioate internucleoside linkages.

In some embodiments, the double-stranded RNA molecule may be in the form of a pharmaceutically acceptable salt. The salt may be a sodium salt or a potassium salt.

The double-stranded RNA molecule may be an isolated double-stranded RNA molecule or a purified double-stranded RNA molecule. In some embodiments the double-stranded RNA molecule of the invention is a manufactured (man made) double-stranded RNA molecule.

The present invention also provides a pharmaceutical composition comprising the double-stranded RNA molecule of the invention and a pharmaceutically acceptable diluent, solvent, carrier, salt and/or adjuvant.

The present invention also provides a method for treating or preventing a disease in a subject comprising administering a therapeutically or prophylactically effective amount of the double-stranded RNA molecule of the invention or the pharmaceutical composition of the invention, to a subject in need thereof.

The present invention also provides the double-stranded RNA molecule of the invention or the pharmaceutical composition of the invention for use as a medicament in the treatment of a disease.

The present invention also provides the use of the double-stranded RNA molecule of the invention or the pharmaceutical composition of the invention, for the preparation of a medicament for the treatment or prevention of a disease.

In some embodiments, the disease may be conjunctivitis, dry eyes or inflammation.

In some embodiments, the double-stranded RNA molecule may be administered to the eye once per day, twice per day, three times per day or more than three times per day.

In some embodiments, the double-stranded RNA molecule may be administered for less than one day or for one day, two days, three days, four days, five days, six days, seven days or more than seven days.

In some embodiments, the double-stranded RNA molecule may be administered for a period of:
(i) one week, two weeks, three weeks, four weeks, five weeks, six weeks or more than six weeks; or
(ii) one month, two months, three months, four months, five months, six months, or more than 6 months; or
(iii) one year, two years, three years, four years, five years or more than five years.

In some embodiments, the double-stranded RNA molecule may be administered to one eye or to both eyes.

The present invention also provides an in vitro method for modulating expression of a target in a cell, the method comprising administering the double-stranded RNA molecule of the invention or the pharmaceutical composition of the invention, to the cell in an effective amount.

### BRIEF DESCRIPTION OF FIGURES

FIG. 1—AHSA1 expression normalized to HPRT in palpebral conjunctiva Rabbit samples, 96 hours after last dosing. 20-25% knockdown in the entire conjunctiva was observed for siRNA molecules comprising the sequence of SEQ ID NO: 3, with superior knockdown with C16-, C22- and Cholesterol-conjugated AHSA1 siRNA compared to naked (i.e. unconjugated SiRNA) siRNA.

FIG. 2: AHSA1 siRNA content in palpebral conjunctiva Rabbit samples, 96 hours after last dosing. Increased content in the conjunctiva was observed for siRNA molecules comprising the sequence of SEQ ID NO: 3, with superior content with C16-, C22- and especially with Cholesterol-conjugated AHA1 siRNA compared to naked (i.e. unconjugated siRNA) siRNA.

FIG. 3. AHSA1 expression normalized to HPRT in bulbar conjunctiva EYEPRIM Rabbit samples, 96 hours after last dosing. 69% knockdown (C16-conjugated SEQ ID NO: 3), 65% knockdown (C22-conjugated SEQ ID NO: 3) and 61% knockdown (Cholesterol-conjugated SEQ ID NO: 3) in the conjunctiva EYEPRIM samples were compared to 4% for naked (i.e. unconjugated siRNA) siRNA (SEQ ID NO: 3 (no conjugation)), significant superior knockdown with C16-(p=0.02 Student's t-test), and superior knockdown with C22- and Cholesterol-conjugated AHSA1 siRNA compared to naked siRNA.

FIG. 4. Biophysical analysis of different fatty acid conjugated AHA-1 specific siRNAs (FA-siRNA). Column 3 shows the propensity of the different fatty acid conjugates to exist in different oligomeric states at 25 μM concentration (Final Oligomeric State as measured by AUC); Column 4 shows monomer percentage of different FA-siRNAs when dissolved in 25 μM PBS; Column 5 shows binding affinity to mouse serum albumin (MSA; determined by ITC); Column 6 shows the number of FA-siRNA conjugates bound to MSA.

FIG. 5. Conjunctival tissue ISH staining was observed for naked siRNA molecules comprising the sequence of SEQ ID NO:3, C16-conjugated siRNA molecules comprising the sequence of SEQ ID NO: 3, C22-conjugated siRNA molecules comprising the sequence of SEQ ID NO: 3 and cholesterol-conjugated siRNA molecules comprising the sequence of SEQ ID NO: 3, indicating superior tissue staining with C16, C22 and Cholesterol AHSA1 siRNA compared to naked AHSA1 siRNA, whereas C16 and C22 AHAS1 siRNA showed the most superior result compared to naked and Cholesterol AHAS1 siRNA. In addition, staining for naked AHSA1 siRNA was mainly located at the superficial conjunctiva whereas staining for C16, C22 and Cholesterol AHSA1 siRNA was also located in the stroma of the conjunctival tissue.

### DETAILED DESCRIPTION OF THE INVENTION

### Nucleic Acid Molecule

The term “nucleic acid molecule” or “therapeutic nucleic acid molecule” as used herein is defined as it is generally understood by the skilled person as a molecule comprising two or more covalently linked nucleosides (i.e. a nucleotide sequence).

The nucleic acid molecule(s) referred to in the invention are generally therapeutic oligonucleotides below 50 nucleotides in length.

As used herein, the terms “polynucleotide”, “nucleotide”, “nucleic acid”, “nucleic acid molecule” and “nucleic acid sequence” are intended to be synonymous with each other.

Nucleic acid molecules are commonly made in the laboratory by solid-phase chemical synthesis followed by purification and isolation. When referring to a sequence of the nucleic acid molecule, reference is made to the sequence or order of nucleobase moieties, or modifications thereof, of the covalently linked nucleotides or nucleosides. The nucleic acid molecule of the invention is man-made, and is chemically synthesized, and is typically purified or isolated. The nucleic acid molecule of the invention may comprise one or more modified nucleosides or nucleotides.

The nucleic acid molecule of the invention may comprise one or more modified nucleosides, such as 2′ sugar modified nucleosides. The nucleic acid molecule of the invention may comprise one or more modified internucleoside linkages, such as one or more phosphorothioate internucleoside linkages.

### Oligonucleotide

The term “oligonucleotide” as used herein is defined, as is generally understood by the skilled person, as a molecule comprising two or more covalently linked nucleosides. Such covalently bound nucleosides may also be referred to as nucleic acid molecules or oligomers.

### Double-Stranded RNA Molecule

Herein, the term “RNA interference (RNAi) molecule”, “RNAi molecule” or “RNAi” refers to a short, typically double-stranded, RNA molecule capable of inducing RNA-dependent gene silencing via the RNA-induced silencing complex (RISC) in a cell's cytoplasm, where they interact with the catalytic RISC component argonaute. One type of RNAi molecule is a small interfering RNA (siRNA), which is a typically double-stranded RNA molecule that, by binding complementary mRNA after transcription, typically leads to degradation of the mRNA and loss in translation. In other words, the term “siRNA molecule” as used herein is defined as a nucleic acid molecule capable of modulating expression of a target by binding to a target nucleic acid, in particular to a contiguous sequence on a target nucleic acid. siRNA molecules are typically 20-24 base pairs in length and usually have phosphorylated 5′ ends and hydroxylated 3′ ends with two overhanging nucleotides. A small interfering RNA (siRNA) may also be known as a short interfering RNA or a silencing RNA.

In some embodiments, the double-stranded RNA molecule of the invention may be a small interfering RNA (siRNA) molecule.

The double-stranded RNA molecule of the invention comprises a first strand having a 5′ end and a 3′ end, and a second strand having a 5′ end and a 3′ end, wherein the first strand is complementary to the second strand.

The double-stranded RNA molecule may be described in terms of comprising a sense strand and an antisense strand.

In some embodiments, the first strand may be a sense strand (i.e. is in sense orientation) and the second strand may be an antisense strand (i.e. is in antisense orientation).

In some embodiments, the first strand may be an antisense strand (i.e. is in antisense orientation) and the second strand may be a sense strand (i.e. is in sense orientation).

Another type of RNAi molecule is a small hairpin RNA (shRNA) which is an artificial RNA molecule with a hairpin structure which, upon expression, is able to reduce the level of a target mRNA via the DICER and RNA reducing silencing complex (RISC). A small hairpin RNA (shRNA) may also be known as a short hairpin RNA. In some embodiments, the double-stranded RNA molecule of the invention may be a small hairpin RNA (shRNA) molecule.

RNAi molecules can be designed on the basis of the RNA sequence of the gene of interest. Corresponding RNAi molecules can then be synthesized chemically or by in vitro transcription, or expressed from a vector or PCR product.

siRNA and shRNA molecules are generally between 20 and 50 nucleotides in length, such as between 25 and 35 nucleotides in length, and may interact with the endonuclease known as Dicer which is believed to processes dsRNA into 19-23 base pair short interfering RNAs (siRNAs) with characteristic two base 3′ overhangs which are then incorporated into an RNA-induced silencing complex (RISC). Effective extended forms of Dicer substrates have been described in U.S. Pat. Nos. 8,349,809 and 8,513,207, hereby incorporated by reference. Upon binding to the appropriate target mRNA, one or more endonucleases within the RISC cleave the target to induce silencing. RNAi agents may be chemically modified using modified internucleotide linkages and high affinity nucleosides, such as 2′-4′ bicyclic ribose modified nucleosides, including LNA and cET.

In some embodiments, the double-stranded RNA molecule of the invention comprises or consists of about 8 to 50 nucleotides in length.

In some embodiments, the double-stranded RNA molecule of the invention comprises or consists of about 12 to 50 nucleotides in length, such as about 15 to 45, such as about 20 to 40, such as about 25 to 35, contiguous nucleotides in length.

In some embodiments, the double-stranded RNA molecule comprises or consists of about 18-25 nucleotides in length. In some embodiments, the double-stranded RNA molecule comprises or consists of about 18-30 nucleotides in length. In some embodiments, the double-stranded RNA molecule comprises or consists of about 18-35 nucleotides in length. In some embodiments, the double-stranded RNA molecule comprises or consists of about 20-25 nucleotides in length. In some embodiments, the double-stranded RNA molecule comprises or consists of about 20-30 nucleotides in length. In some embodiments, the double-stranded RNA molecule comprises or consists of about 20-35 nucleotides in length. It is to be understood that any range given herein includes the range endpoints. Accordingly, if a double-stranded RNA molecule is said to include from about 10 to 30 nucleotides, lengths of both about 10 and about 30 nucleotides are included.

In some embodiments, the double-stranded RNA molecule comprises or consists of about 50 or fewer nucleotides, about 45 or fewer nucleotides, about 40 or fewer nucleotides, about 35 or fewer nucleotides, about 30 or fewer nucleotides, about 25 or fewer nucleotides, about 20 or fewer nucleotides, or about 15 or fewer nucleotides.

In some embodiments, the double-stranded RNA molecule comprises or consists of about 10 or more nucleotides, about 15 or more nucleotides, about 20 or more nucleotides, about 25 or more nucleotides, about 30 or more nucleotides, about 35 or more nucleotides, about 40 or more nucleotides, or about 45 or more nucleotides.

In some embodiments, the double-stranded RNA molecule comprises or consists of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

In some embodiments, the double-stranded RNA molecule may be at least about 20 nucleotides in length. The double-stranded RNA molecule may be 20, 21, 22, 23 or 24 nucleotides in length.

In some embodiments, the double-stranded RNA molecule comprises or consists of 18 nucleotides in length.

In some embodiments, the double-stranded RNA molecule comprises or consists of 19 nucleotides in length.

In some embodiments, the double-stranded RNA molecule comprises or consists of 20 nucleotides in length.

In some embodiments, the double-stranded RNA molecule comprises or consists of 21 nucleotides in length.

In some embodiments, the double-stranded RNA molecule comprises or consists of 22 nucleotides in length.

In some embodiments, the double-stranded RNA molecule comprises or consists of 23 nucleotides in length.

In some embodiments, the double-stranded RNA molecule comprises or consists of 24 nucleotides in length.

In some embodiments, the double-stranded RNA molecule comprises or consists of 25 nucleotides in length.

In some embodiments, the double-stranded RNA molecule comprises or consists of 26 nucleotides in length.

In some embodiments, the double-stranded RNA molecule comprises or consists of 27 nucleotides in length.

In some embodiments, the double-stranded RNA molecule comprises or consists of 28 nucleotides in length.

In some embodiments, the double-stranded RNA molecule comprises or consists of 29 nucleotides in length.

In some embodiments, the double-stranded RNA molecule comprises or consists of 30 nucleotides in length.

In some embodiments, the double-stranded RNA molecule comprises or consists of 31 nucleotides in length.

In some embodiments, the double-stranded RNA molecule comprises or consists of 32 nucleotides in length.

In some embodiments, the double-stranded RNA molecule comprises or consists of 33 nucleotides in length.

In some embodiments, the double-stranded RNA molecule comprises or consists of 34 nucleotides in length.

In some embodiments, the double-stranded RNA molecule comprises or consists of 35 nucleotides in length.

The double-stranded RNA molecule(s) binds to a target nucleic acid expressed in the eye of an animal, in particular the eye of a mammal. In some embodiments, the double-stranded RNA molecule is typically for inhibiting and/or modulating the expression of a target nucleic acid sequence expressed in the eye.

### Contiguous Nucleotide Sequence

The term “contiguous nucleotide sequence” refers to the region of the double-stranded RNA molecule which is complementary to a target nucleic acid, which may be or may comprise an oligonucleotide motif sequence. The term is used interchangeably herein with the term “contiguous nucleobase sequence”.

One of the strands of the double-stranded RNA molecule comprises or consists of the contiguous nucleotide sequence. In some embodiments, the first strand comprises or consists of the contiguous nucleotide sequence. In some embodiments, the second strand comprises or consists of the contiguous nucleotide sequence.

In some embodiments, the strand of the double-stranded RNA molecule that comprises or consists of the contiguous nucleotide sequence may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid.

It is understood that the contiguous nucleotide sequence cannot be longer than the double-stranded RNA molecule (or strands thereof) as such, and that the double-stranded RNA molecule (or strands thereof) cannot be shorter than the contiguous nucleotide sequence.

In some embodiments, all of the nucleosides of the first or second strand of the double-stranded RNA molecule may constitute the contiguous nucleotide sequence.

The contiguous nucleotide sequence is the sequence of nucleotides in the first or second strand of the double-stranded RNA molecule of the invention which are complementary to, and in some instances fully complementary to, the target nucleic acid, target sequence, or target site sequence.

In some embodiments, the contiguous nucleotide sequence is about 8 to 50 nucleotides in length.

In some embodiments, the contiguous nucleotide sequence may comprise or consist of about 12 to 50 nucleotides in length, such as from about 15 to 45, such as from about 20 to 40, such as from about 25 to 35, contiguous nucleotides in length.

In some embodiments, the contiguous nucleotide sequence comprises or consists of about 18-25 nucleotides in length. In some embodiments, the contiguous nucleotide sequence comprises or consists of about 18-30 nucleotides in length. In some embodiments, the contiguous nucleotide sequence comprises or consists of about 18-35 nucleotides in length.

In some embodiments, the contiguous nucleotide sequence comprises or consists of about 20-25 nucleotides in length. In some embodiments, the contiguous nucleotide sequence comprises or consists of about 20-30 nucleotides in length. In some embodiments, the contiguous nucleotide sequence comprises or consists of about 20-35 nucleotides in length. It is to be understood that any range given herein includes the range endpoints. Accordingly, if a contiguous nucleotide sequence is said to include from about 10 to 30 nucleotides, both about 10 and about 30 nucleotides are included.

In some embodiments, the contiguous nucleotide sequence may comprise or consist of about 50 or fewer nucleotides, about 45 or fewer nucleotides, about 40 or fewer nucleotides, about 35 or fewer nucleotides, about 30 or fewer nucleotides, about 25 or fewer nucleotides, about 20 or fewer nucleotides, or about 15 or fewer nucleotides.

In some embodiments, the contiguous nucleotide sequence may comprise or consist of about 10 or more nucleotides, about 15 or more nucleotides, about 20 or more nucleotides, about 25 or more nucleotides, about 30 or more nucleotides, about 35 or more nucleotides, about 40 or more nucleotides, or about 45 or more nucleotides.

In some embodiments, the contiguous nucleotide sequence comprises or consists of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

In some embodiments, the contiguous nucleotide sequence may be at least about 20 nucleotides in length. The contiguous nucleotide sequence may be 20, 21, 22, 23 or 24 nucleotides in length.

In some embodiments, the contiguous nucleotide sequence comprises or consists of 18 nucleotides in length.

In some embodiments, the contiguous nucleotide sequence comprises or consists of 19 nucleotides in length.

In some embodiments, the contiguous nucleotide sequence comprises or consists of 20 nucleotides in length.

In some embodiments, the contiguous nucleotide sequence comprises or consists of 21 nucleotides in length.

In some embodiments, the contiguous nucleotide sequence comprises or consists of 22 nucleotides in length.

In some embodiments, the contiguous nucleotide sequence comprises or consists of 23 nucleotides in length.

In some embodiments, the contiguous nucleotide sequence comprises or consists of 24 nucleotides in length.

In some embodiments, the contiguous nucleotide sequence comprises or consists of 25 nucleotides in length.

In some embodiments, the contiguous nucleotide sequence comprises or consists of 26 nucleotides in length.

In some embodiments, the contiguous nucleotide sequence comprises or consists of 27 nucleotides in length.

In some embodiments, the contiguous nucleotide sequence comprises or consists of 28 nucleotides in length.

In some embodiments, the contiguous nucleotide sequence comprises or consists of 29 nucleotides in length.

In some embodiments, the contiguous nucleotide sequence comprises or consists of 30 nucleotides in length.

In some embodiments, the contiguous nucleotide sequence comprises or consists of 31 nucleotides in length.

In some embodiments, the contiguous nucleotide sequence comprises or consists of 32 nucleotides in length.

In some embodiments, the contiguous nucleotide sequence comprises or consists of 33 nucleotides in length.

In some embodiments, the contiguous nucleotide sequence comprises or consists of 34 nucleotides in length.

In some embodiments, the contiguous nucleotide sequence comprises or consists of 35 nucleotides in length.

In some embodiments, the contiguous nucleotide sequence is the same length as the first and/or second strand of the double-stranded RNA molecule.

In some embodiments, the first or second strand of the double-stranded RNA molecule consists of the contiguous nucleotide sequence.

In some embodiments, the first or second strand of the double-stranded RNA molecule is the contiguous nucleotide sequence.

### Conjugate Moiety

The inventors have identified that double-stranded RNA molecules capable of binding to a target sequence can be effectively administered to the eye by conjugating the double-stranded RNA molecule to at least one conjugate moiety.

As is illustrated in the examples, the administration of the double-stranded RNA molecule of the invention to the eye can inhibit the expression of a target, in particular a target expressed in the eye.

The double-stranded RNA molecule of the invention is attached to at least one conjugate moiety. In some embodiments the double-stranded RNA molecule may be attached to more than one conjugate moiety.

In some embodiments the conjugate moiety or conjugate moieties may be referred to as a conjugate of the invention. In some embodiments, the double-stranded RNA molecule is covalently attached to at least one conjugate moiety.

The terms “attached”, “positioned”, “linked” and “conjugated” are interchangeable in reference to the double-stranded RNA molecule and the conjugate moiety.

The term “conjugate” as used herein refers to a double-stranded RNA molecule which is linked, such as covalently linked, to a conjugate moiety. The conjugate moiety may be linked, such as covalently linked, to the double-stranded RNA molecule directly, or the conjugate moiety may be linked to the double-stranded RNA molecule via a linker group.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the conjugate moiety is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) and combinations thereof.

In some embodiments, the conjugate moiety may be a fatty acid molecule. In some embodiments, the conjugate moieties may be fatty acid molecules.

A “fatty acid” is a molecule that typically consists of a chain of carbon atoms, typically with hydrogen atoms bonded to the carbon atoms along the length of the chain. In other words, a fatty acid molecule comprises hydrocarbons. Hydrogen atoms are also typically found at one end (or terminus) of the chain of the fatty acid molecule and a carboxyl group (—COOH) is typically found at the other end (or terminus) of the chain. Indeed, it is the carboxyl group that makes the molecule an acid (e.g. a carboxylic acid). The terms “fatty acid” and “fatty acid molecule” as used herein are considered to be interchangeable.

The term “fatty acid” encompasses one single fatty acid molecule as well as a mixture of two or more fatty acid molecules, such as two or more different fatty acid molecules.

In embodiments where more than one fatty acid molecule is attached to the double-stranded RNA molecule, each fatty acid molecule is independently selected such that the fatty acid molecules attached to the double-stranded RNA molecule may or may not be the same.

In some embodiments, two or more fatty acids may be connected with a linker. It will be understood that the linker connecting the two or more fatty acids may attach to any point on each of the two or more fatty acid molecules.

The fatty acid molecule may be a molecule comprised of carbon atoms. The fatty acid molecule may have the formula CX, where C means carbon and X refers to the total number of carbon atoms (e.g. the carbon chain length) that are present in the fatty acid molecule (e.g. “C12” refers to a fatty acid molecule of 12 carbons).

In some embodiments, the fatty acid molecule may be a molecule with 3-40 carbon atoms (e.g. C3-C40).

In some embodiments, the fatty acid molecule is selected from the list consisting of: C3, C4, C5, C6, C7, C8, C9, C10, C11, C12, C13, C14, C15, C16, C17, C18, C19, C20, C21, C22, C23, C24, C25, C26, C27, C28, C29, C30, C31, C32, C33, C34, C35, C36, C37, C38, C39 and C40.

In some embodiments, the fatty acid molecule may be a molecule with 12-24 carbon atoms (e.g. C12-C24).

In some embodiments, the fatty acid molecule is selected from the list consisting of: C12, C13, C14, C15, C16, C17, C18, C19, C20, C21, C22, C23 and C24.

In some embodiments, the fatty acid molecule is C10.

In some embodiments, the fatty acid molecule is C16.

In some embodiments, the fatty acid molecule is C18.

In some embodiments, the fatty acid molecule is C22.

In some embodiments, the fatty acid molecule may be a salt of a fatty acid molecule (e.g. a fatty acid salt).

In some embodiments, the fatty acid molecule may be branched or unbranched. In other words, the fatty acid molecule may comprise branched chains or may comprise unbranched chains. It will be understood that “unbranched” refers to a linear chain of atoms, for example, a linear chain of carbon atoms bonded to hydrogen atoms (e.g. CH2 groups), where the carbon atoms are also linked by carbon-carbon bonds.

It will also be understood that “branched” refers to a non-linear chain of atoms, for example, where one or more carbon groups form a branch by attaching to another chain of carbon atoms bonded to hydrogen atoms (e.g. CH2 groups), where the carbon atoms are also linked by carbon-carbon bonds. For example, a branched chain fatty acid molecule may comprise one or more carbon groups (such as methyl group(s)) attached to a chain of carbon atoms. The one or more carbon group(s) forming the branch(es) may be attached at any point along the carbon chain (i.e. attached to one or more of position C2 to CN-1, where C means carbon and N refers to the total number of carbon atoms in a linear chain).

It will be understood that where two or more fatty acids are connected by a linker, all of the fatty acids may be branched. It will also be understood that where two or more fatty acids are connected by a linker, all of the fatty acids may be unbranched. It will also be understood that where two or more fatty acids are connected by a linker, one or more of the fatty acids may be branched and the remaining one or more fatty acids may be unbranched.

It will be understood that a fatty acid which does not comprise a carbon double bond (e.g. a C═C bond) may be referred to as a saturated fatty acid. It will be understood that the term “saturated” means that the maximum possible number of atoms (e.g. hydrogen atoms) are bonded to each carbon in the molecule

In some embodiments, the fatty acid molecule may be a saturated fatty acid. In some embodiments, the fatty acid molecule may be fully saturated (i.e. comprising carbon single bonds but not comprising a carbon double bond or a carbon triple bond). In some embodiments, the fatty acid molecule may be partially saturated (i.e. comprising a combination of (i) one or more carbon single bond(s); and (ii) one or more carbon non-single bond(s) (i.e. carbon double bond(s) and/or carbon triple bond(s))).

In some embodiments, the fatty acid molecule may comprise a carbon double bond (e.g. a C═C bond). In some embodiments, the fatty acid molecule may comprise a carbon triple bond (e.g. a C═C bond). In some embodiments, the fatty acid molecule may comprise a combination of a carbon double bond (e.g. a C═C bond) and a carbon triple bond (e.g. a C═C bond). It will be understood that when the fatty acid molecule comprises a carbon double bond(s) and/or a carbon triple bond(s), the remaining bonds in the fatty acid molecule may be carbon single bonds (e.g. C—C bonds).

It will be understood that a fatty acid which comprises one or more carbon double bond(s) and/or one or more carbon triple bond(s) may be referred to as an unsaturated fatty acid. An unsaturated fatty acid comprising one carbon double bond or one carbon triple bond may be referred to as a monounsaturated fatty acid. An unsaturated fatty acid comprising two or more non-single bonds (i.e. carbon double bonds and/or carbon triple bonds) may be referred to as a polyunsaturated fatty acid.

In some embodiments, the fatty acid molecule may be an unsaturated fatty acid. In some embodiments, the fatty acid molecule may be a monounsaturated fatty acid. In some embodiments, the fatty acid molecule may be a polyunsaturated fatty acid.

In some embodiments, the fatty acid molecule may be fully unsaturated (i.e. comprising carbon double bonds and/or carbon triple bonds but not comprising carbon single bonds). In some embodiments, the fatty acid molecule may be partially unsaturated (i.e. comprising a combination of (i) one or more carbon non-single bond(s) (i.e. carbon double bond(s) and/or carbon triple bond(s)); and (ii) one or more carbon single bond(s)).

It will be understood that where two or more fatty acids are connected by a linker, all of the fatty acids may be saturated. It will also be understood that where two or more fatty acids are connected by a linker, all of the fatty acids may be unsaturated. It will also be understood that where two or more fatty acids are connected by a linker, one or more of the fatty acids may be saturated and the remaining one or more fatty acids may be unsaturated.

It will be understood that the saturated or unsaturated fatty acid molecule can be branched or unbranched.

In some embodiments, the saturated fatty acid molecule may be branched or unbranched.

In some embodiments, the unsaturated fatty acid molecule may be branched or unbranched. In some embodiments, the monounsaturated fatty acid molecule may be branched or unbranched. In some embodiments, the polyunsaturated fatty acid molecule may be branched or unbranched.

In some embodiments, the branched fatty acid molecule may be fully saturated, fully unsaturated, or may be a mix of both saturated and unsaturated. In some embodiments, the unbranched fatty acid molecule may be fully saturated, fully unsaturated, or may be a mix of both saturated and unsaturated.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or about 100% of the fatty acid molecule comprises double bonds.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or about 100% of the fatty acid molecule comprises triple bonds.

In some embodiments, the fatty acid molecule comprises one or more double bond(s). In some embodiments, the fatty acid molecule comprises one or more triple bond(s). In some embodiments, the fatty acid molecule comprises a combination of one or more carbon double bond(s) and one or more carbon triple bond(s). It will be understood that when the fatty acid molecule comprises one or more carbon double bond(s) and/or carbon triple bond(s), the remaining bonds in the fatty acid molecule may be carbon single bonds (e.g. C—C bonds).

In some embodiments, the fatty acid molecule comprises two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more carbon double bonds.

In some embodiments, the fatty acid molecule comprises two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more carbon triple bonds.

In some embodiments, the fatty acid molecule comprises a combination of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more carbon double bonds and carbon triple bonds.

It will be understood that more than nineteen carbon double bonds can be used in the present invention. It will be understood that more than nineteen carbon triple bonds can be used in the present invention.

The carbon double bonds may be in cis configuration or in trans configuration. The carbon triple bonds may be in cis configuration or in trans configuration. It will be understood that a cis configuration means that the two function groups (e.g. hydrogen atoms) adjacent to the carbon double bond or carbon triple bond stick out on the same side of the chain, and that a trans configuration means that the two function groups (e.g. hydrogen atoms) adjacent to the carbon double bond or carbon triple bond are on opposite sides of the chain.

In some embodiments, the fatty acid molecule may be a cis-unsaturated fatty acid molecule.

In some embodiments, the fatty acid molecule may be a trans-unsaturated fatty acid molecule.

In some embodiments, the fatty acid molecule may comprise a combination of a cis-unsaturated fatty acid component and a trans-unsaturated fatty acid component. In some embodiments, the fatty acid molecule may comprise a combination of cis and trans double bonds. In some embodiments, the fatty acid molecule may comprise a combination of cis and trans triple bonds. In some embodiments, the fatty acid molecule may comprise a combination of cis and trans double bonds and cis and trans triple bonds.

The fatty acid molecule may have the formula CX:Y, where C means carbon, where X refers to the total number of carbon atoms (i.e. the carbon chain length) that are present in the fatty acid molecule, and where Y refers to the total number of unsaturated/non-single carbon bonds (i.e. carbon double bonds and/or carbon triple bonds) present in the fatty acid molecule (e.g. “C16:5” refers to a fatty acid molecule of 16 carbons in length with a combined total of 5 unsaturated/non-single carbon bonds in total (i.e. 5 carbon double bonds could be present or 5 carbon triple bonds could be present or a combined mix of carbon double bonds and carbon triple bonds totalling 5 non-single bonds could be present). In other words, the number X before the colon specifies the number of carbon atoms and the number Y after the colon specifies the total number of unsaturated/non-single bonds in the fatty acid molecule (i.e. double and/or triple bonds). X can be any positive natural number (i.e. 1, 2, 3, etc.). X cannot be 0 or a negative value. Y can be any non-negative number (i.e. 0, 1, 2, 3, etc).

However, for a given X number (in CX:Y), the maximum number Y of double and/or triple bonds equals X/2-1 (i.e. Y=(X/2)-1). In other words, for a given X number of carbon in the fatty acid molecule, the number Y of double and/or triple bonds can be any non-negative number (i.e. 0, 1, 2, 3, etc) with a maximum of Y=(X/2)-1. Hence, in some embodiments where the fatty acid molecule has the formula CX:Y, Y may equal the maximum value of X/2-1 (i.e. Y=(X/2)-1). In the case of X being an odd number, the result (being a half number) must be rounded down to the nearest natural number. For example, with X=7, the result of (X/2)-1 is 2,5, which must be rounded down to 2. In other words, for X=7, the maximum number of double and/or triple bonds is Y=2; for X=9, the maximum number of double and/or triple bonds is Y-3, etc. In the case of X being an even number, the result of Y do not need to be rounded down: if X=8, then Y=3; if X=10, then Y=4, etc. This means that for X-8 or 9 (i.e. for a fatty acid molecule having 8 or 9 carbons), the maximum number of double and/or triple bonds possible is 3; and that the Y value in the annotation C8:Y or C9:Y can be any natural number from 0 to 3 (i.e. 0, 1, 2 or 3). In other words, for X=8, the possible fatty acids are C8:0, C8:1, C8:2 and C8:3; and for X=9, the possible fatty acids are C9:0, C9:1, C9:2 and C9:3. The same applies for any other X value (X being a positive natural number, i.e. non-null and non-negative).

In some embodiments, the fatty acid molecule is selected from the list consisting of: C3:0; C4:0; C4:1; C5:0; C5:1; C6:0; C6:1; C6:2; C7:0; C7:1; C7:2; C8:0; C8:1; C8:2; C8:3; C9:0; C9:1; C9:2; C9:3; C10:0; C10:1; C10:2; C10:3; C10:4; C11:0; C11:1; C11:2; C11:3; C11:4; C12:0; C12:1; C12:2; C12:3; C12:4; C12:5; C13:0; C13:1; C13:2; C13:3; C13:4; C13:5; C14:0; C14:1; C14:2; C14:3; C14:4; C14:5; C14:6; C15:0; C15:1; C15:2; C15:3; C15:4; C15:5; C15:6; C16:0; C16:1; C16:2; C16:3; C16:4; C16:5; C16:6; C16:7; C17:0; C17:1; C17:2; C17:3; C17:4; C17:5; C17:6; C17:7; C18:0; C18:1; C18:2; C18:3; C18:4; C18:5; C18:6; C18:7; C18:8; C19:0; C19:1; C19:2; C19:3; C19:4; C19:5; C19:6; C19:7; C19:8; C20:0; C20:1; C20:2; C20:3; C20:4; C20:5; C20:6; C20:7; C20:8; C20:9; C21:0; C21:1; C21:2; C21:3; C21:4; C21:5; C21:6; C21:7; C21:8; C21:9; C22:0; C22:1; C22:2; C22:3; C22:4; C22:5; C22:6; C22:7; C22:8; C22:9; C22:10; C23:0; C23:1; C23:2; C23:3; C23:4; C23:5; C23:6; C23:7; C23:8; C23:9; C23:10; C24:0; C24:1; C24:2; C24:3; C24:4; C24:5; C24:6; C24:7; C24:8; C24:9; C24:10; C24:11; C25:0; C25:1; C25:2; C25:3; C25:4; C25:5; C25:6; C25:7; C25:8; C25:9; C25:10; C25:11; C26:0; C26:1; C26:2; C26:3; C26:4; C26:5; C26:6; C26:7; C26:8; C26:9; C26:10; C26:11; C26:12; C27:0; C27:1; C27:2; C27:3; C27:4; C27:5; C27:6; C27:7; C27:8; C27:9; C27:10; C27:11; C27:12; C28:0; C28:1; C28:2; C28:3; C28:4; C28:5; C28:6; C28:7; C28:8; C28:9; C28:10; C28:11; C28:12; C28:13; C29:0; C29:1; C29:2; C29:3; C29:4; C29:5; C29:6; C29:7; C29:8; C29:9; C29:10; C29:11; C29:12; C29:13; C30:0; C30:1; C30:2; C30:3; C30:4; C30:5; C30:6; C30:7; C30:8; C30:9; C30:10; C30:11; C30:12; C30:13; C30:14; C31:0; C31:1; C31:2; C31:3; C31:4; C31:5; C31:6; C31:7; C31:8; C31:9; C31:10; C31:11; C31:12; C31:13; C31:14; C32:0; C32:1; C32:2; C32:3; C32:4; C32:5; C32:6; C32:7; C32:8; C32:9; C32:10; C32:11; C32:12; C32:13; C32:14; C32:15; C33:0; C33:1; C33:2; C33:3; C33:4; C33:5; C33:6; C33:7; C33:8; C33:9; C33:10; C33:11; C33:12; C33:13; C33:14; C33:15; C34:0; C34:1; C34:2; C34:3; C34:4; C34:5; C34:6; C34:7; C34:8; C34:9; C34:10; C34:11; C34:12; C34:13; C34:14; C34:15; C34:16; C35:0; C35:1; C35:2; C35:3; C35:4; C35:5; C35:6; C35:7; C35:8; C35:9; C35:10; C35:11; C35:12; C35:13; C35:14; C35:15; C35:16; C36:0; C36:1; C36:2; C36:3; C36:4; C36:5; C36:6; C36:7; C36:8; C36:9; C36:10; C36:11; C36:12; C36:13; C36:14; C36:15; C36:16; C36:17; C37:0; C37:1; C37:2; C37:3; C37:4; C37:5; C37:6; C37:7; C37:8; C37:9; C37:10; C37:11; C37:12; C37:13; C37:14; C37:15; C37:16; C37:17; C38:0; C38:1; C38:2; C38:3; C38:4; C38:5; C38:6; C38:7; C38:8; C38:9; C38:10; C38:11; C38:12; C38:13; C38:14; C38:15; C38:16; C38:17; C38:18; C39:0; C39:1; C39:2; C39:3; C39:4; C39:5; C39:6; C39:7; C39:8; C39:9; C39:10; C39:11; C39:12; C39:13; C39:14; C39:15; C39:16; C39:17; C39:18; C40:0; C40:1; C40:2; C40:3; C40:4; C40:5; C40:6; C40:7; C40:8; C40:9; C40:10; C40:11; C40:12; C40:13; C40:14; C40:15; C40:16; C40:17; C40:18; C40:19

The carbon double bond may be positioned at any point (i.e. between any pair of adjacent carbon atoms) in the fatty acid molecule. The carbon double bonds may be positioned at any points (i.e. between multiple pairs of adjacent carbon atoms) in the fatty acid molecule. The carbon triple bond may be positioned at any point (i.e. between any pair of adjacent carbon atoms) in the fatty acid molecule. The carbon triple bonds may be positioned at any points (i.e. between multiple pairs of adjacent carbon atoms) in the fatty acid molecule.

In some embodiments, the fatty acid molecule may include one or more modifications and/or substitutions. In some embodiments, the fatty acid molecule may include one or more amino acids. In some embodiments, the fatty acid molecule may include one or more sugar or carbohydrate molecules. It will be understood that modifications and/or substitutions may be made to saturated, unsaturated, monounsaturated and polyunsaturated fatty acid molecules. It will also be understood that modifications and/or substitutions may be made to branched and unbranched fatty acid molecules.

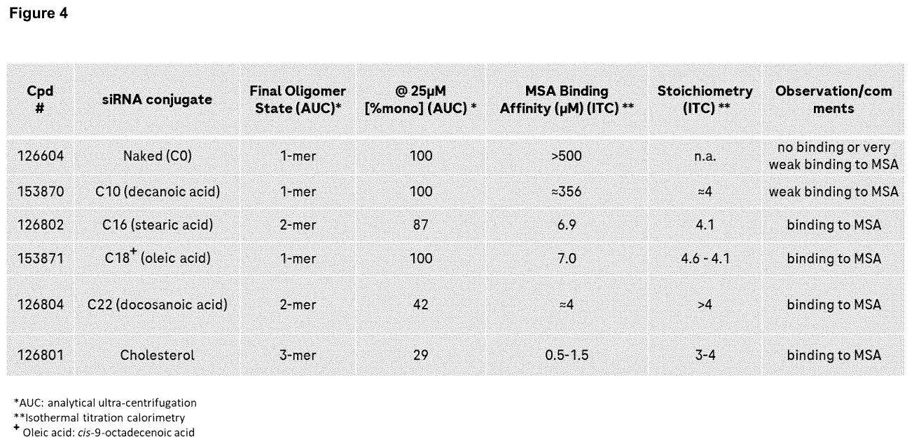

It will be understood that, as shown in FIG. 4, the binding strength of the double-stranded RNA molecule of the invention may be purposefully controlled (e.g. tuned) by the length of the fatty acid molecule/moiety used. Without wishing to be bound by theory, it may be considered that the binding strength of the double-stranded RNA molecule of the invention (i.e. conjugated to a fatty acid molecule) may be proportional to the length of the fatty acid, as exemplified in FIG. 4 with the C10, C16 and C22 fatty acid conjugates, in particular where albumin is used as a transport vehicle.

In some embodiments, the fatty acid molecule/moiety used as the conjugate moiety may be chosen based on the required binding strength according to the needs of the particular intended use of the double-stranded RNA molecule of the invention.

In some embodiments, the double-stranded RNA molecule of the invention may be administered in combination with albumin. The albumin may be serum albumin, such as mouse serum albumin or human serum albumin.

Without wishing to be bound by theory, binding of the double-stranded RNA molecule of the invention to albumin may be one of the mechanisms by which the double-stranded RNA molecule of the invention can be transported, e.g. into a cell.

Without wishing the be bound by theory, the binding strength of the double-stranded RNA molecule of the invention can be exploited to influence (i) the circulation profile of the double-stranded RNA of the invention in plasma; (ii) the stability of the double-stranded RNA of the invention in biological fluids (e.g. tear fluid); and (iii) the intracellular uptake of the double-stranded RNA of the invention into disease relevant tissues.

In some embodiments, the fatty acid molecule of the double-stranded RNA molecule of the invention may be a fatty acid molecule of a suitable binding strength to albumin to influence the circulation profile of the double-stranded RNA of the invention in plasma. In some embodiments, the suitable binding strength may be a binding strength suitable for binding to albumin.

In some embodiments, the fatty acid molecule conjugated to the double-stranded RNA of the invention may be a fatty acid molecule of a suitable binding strength to albumin to influence the stability of the double-stranded RNA of the invention in biological fluids (e.g. tear fluid). In some embodiments, the suitable binding strength may be a binding strength suitable for binding to albumin.

In some embodiments, the fatty acid molecule conjugated to the double-stranded RNA of the invention may be a fatty acid molecule of a suitable binding strength to albumin to influence the intracellular uptake of the double-stranded RNA of the invention into disease relevant tissues. In some embodiments, the suitable binding strength may be a binding strength suitable for binding to albumin.

In some embodiments, the conjugate moiety is a cholesterol molecule. In some embodiments, the conjugate moieties may be cholesterol molecules. It will be understood that the term “cholesterol molecule” and “cholesterol moiety” are interchangeable.

In embodiments where more than one cholesterol molecule is attached to the double-stranded RNA molecule, each cholesterol molecule is independently selected such that the cholesterol molecule attached to the double-stranded RNA molecule may or may not be the same.

In some embodiments, two or more cholesterol molecules may be connected with a linker. It will be understood that the linker connecting the two or more cholesterol molecules may attach to any point on each of the two or more cholesterol molecules.

In some embodiments, the cholesterol moiety is selected from the group comprising: 3′-cholesteryl-TEG CPG, 5′-cholesterol-TEG-CE phosphoramidite, 5′-cholesterol-CE phosphoramidite or cholesteryl-TEG-CE phosphoramidite (TEG=triethyleneglycol, CPG=Controlled Pore Glass Synthesis Supports aka CPG support).

In some embodiments, the cholesterol moiety is 3′-cholesteryl-TEG CPG. In some embodiments, the cholesterol moiety is derived from 3′-cholesteryl-TEG CPG. For example, 3′-cholesteryl-TEG CPG can be used as an agent to introduce a cholesterol moiety at the 3′ end of one of the strands of the double-stranded RNA molecule.

In some embodiments, the cholesterol moiety is 5′-cholesterol-TEG-CE phosphoramidite. In some embodiments, the cholesterol moiety is derived from 5′-cholesterol-TEG-CE phosphoramidite. For example, 5′-cholesterol-TEG-CE phosphoramidite can be used as an agent to introduce a cholesterol moiety at the 5′ end of one of the strands of the double-stranded RNA molecule.

In some embodiments, the cholesterol moiety is 5′-cholesterol-CE phosphoramidite. In some embodiments, the cholesterol moiety is derived from 5′-cholesterol-CE phosphoramidite. For example, 5′-cholesterol-CE phosphoramidite can be used as an agent to introduce a cholesterol moiety at the 5′ end of one of the strands of the double-stranded RNA molecule.

In some embodiments, the cholesterol moiety is cholesteryl-TEG-CE phosphoramidite. In some embodiments, the cholesterol moiety is derived from cholesteryl-TEG-CE phosphoramidite. For example, cholesteryl-TEG-CE phosphoramidite can be used as an agent to introduce a cholesterol moiety at the end of one of the strands of the double-stranded RNA molecule.

In some embodiments, the strands of the double-stranded RNA molecule can by synthesized by using regular amidites or inverted amidites.

In some embodiments, the cholesterol molecule or cholesterol moiety is attached at the 3′ end of one of the strands of the double-stranded RNA molecule by using inverted amidites (e.g. nucleoside phosphoramidites). In other words, cholesterol can be attached to the 3′ end of one of the strands by building the strand using inverted amidites followed by use of any 5′ cholesterol amidites in the end of the synthesis, which will lead to the cholesterol being positioned in the 3′ end of the strand.

In some embodiments, the conjugate moiety (e.g. the fatty acid molecule or the cholesterol molecule) is positioned at the 5′ end or the 3′ end of one of the strands of the double-stranded RNA molecule.

In some embodiments, the conjugate moiety is positioned at the 5′ end of the first strand of the double-stranded RNA molecule. In some embodiments, the conjugate moiety is positioned at the 3′ end of the first strand of the double-stranded RNA molecule.

In some embodiments, the conjugate moiety is positioned at the 5′ end of the second strand of the double-stranded RNA molecule. In some embodiments, the conjugate moiety is positioned at the 3′ end of the second strand of the double-stranded RNA molecule.

The double-stranded RNA molecule may be described in terms of comprising a sense strand and an antisense strand.

In some embodiments, the conjugate moiety is positioned at the 5′ end of the sense strand of the double-stranded RNA molecule. In some embodiments, the conjugate moiety is positioned at the 3′ end of the sense strand of the double-stranded RNA molecule.

In some embodiments, the conjugate moiety is positioned at the 5′ end of the antisense strand of the double-stranded RNA molecule. In some embodiments, the conjugate moiety is positioned at the 3′ end of the antisense strand of the double-stranded RNA molecule.

In some embodiments, the conjugate moiety is not positioned at an end terminal position of either strand (i.e. the conjugate moiety is not positioned at the 5′ end or the 3′ end of either strand). For example, the conjugate moiety may be attached to a position in the middle or center region of the contiguous nucleotide sequence. Herein the terms “middle” and “center” are intended to indicate that the conjugate moiety is not located at either end of the strand, and not that the conjugate moiety is position equidistant from each end.

In some embodiments, the conjugate moiety is positioned at any position of the contiguous nucleotide sequence. In some embodiments, the conjugate moiety is positioned at any position on the double-stranded RNA molecule.

### Linkers

A linkage, linker or spacer is a connection between two atoms that links one chemical group or segment of interest to another chemical group or segment of interest via one or more covalent bonds. Conjugate moieties can be attached to the double-stranded RNA molecule directly or through a linking moiety (e.g. linker or spacer). The linkers serve to covalently connect a conjugate moiety to a double-stranded RNA molecule or contiguous nucleotide sequence thereof.

Used herein, the terms “linker” and “spacer” are interchangeable.

In some embodiments of the invention, the double-stranded RNA molecule of the invention may comprise a linker (also termed “linker region”) which is positioned between the double-stranded RNA molecule and the conjugate moiety. The linker may be attached to the contiguous nucleotide sequence of a strand of the double-stranded RNA molecule complementary to the target nucleic acid and the conjugate moiety.

In some embodiments, the linker is C6.

In some embodiments, the linker is TEG.

In some embodiments, the linker is a dinucleotide. In some embodiments, the linker is a CA dinucleotide.

In some embodiments, the linker is a biocleavable linker. Biocleavable linkers comprise or consist of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In some embodiments, the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

In some embodiments, the linker is not a biocleavable linker. Linkers that are not necessarily biocleavable but primarily serve to covalently connect a conjugate moiety to an oligonucleotide are known. These linkers may comprise a chain structure or an oligomer of repeating units such as ethylene glycol, amino acid units or amino alkyl groups, or a combination thereof. In some embodiments, the linker is an amino alkyl, such as a C2-C36 amino alkyl group, including, for example C6 to C12 amino alkyl groups.

Additional 5′ and/or 3′ Nucleosides

In some embodiments, one or both of the strands of the double-stranded RNA molecule of the invention may further comprise additional 5′ and/or 3′ nucleosides. In other words, in some embodiments, the double-stranded RNA molecule of the invention may comprise 5′ and/or 3′ nucleosides in addition to the contiguous nucleotide sequence.

The further 5′ and/or 3′ nucleosides may or may not be complementary, such as fully complementary, to the target nucleic acid.

The addition of the further 5′ and/or 3′ nucleosides may be used for the purpose of joining the contiguous nucleotide sequence to a conjugate moiety or another functional group. When used for joining the contiguous nucleotide sequence with a conjugate moiety it can serve as a biocleavable linker. Alternatively, it may be used to provide exonucleoase protection or for ease of synthesis or manufacture.

The further 5′ and/or 3′ nucleosides may independently comprise or consist of 1, 2, 3, 4, 5 or more than 5 additional nucleotides, which may be complementary or non-complementary to the target nucleic acid.

The further 5′ and/or 3′ nucleosides may serve as a nuclease susceptible biocleavable linker.

In some embodiments the additional 5′ and/or 3′ end nucleotides are linked with phosphodiester linkages, and are DNA or RNA. Nucleotide based biocleavable linkers suitable for such use are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In some embodiments the internucleoside linkage positioned between the additional 5′ and/or 3′ nucleosides and the contiguous nucleotide sequence is a phosphodiester linkage.

### Administration to the Eye

The double-stranded RNA molecule of the invention is topically administered to the eye (e.g. ophthalmic administration).

It is to be understood that the double-stranded RNA molecule of the invention can be administered to any part of the eye.

In some embodiments, the double-stranded RNA molecule is for administration to the front of the eye.

The conjunctiva and the cornea are both components of the eye. In some embodiments, the double-stranded RNA molecule is for administration to the conjunctiva. In some embodiments, the double-stranded RNA molecule is for administration to the cornea. In some embodiments, the double-stranded RNA molecule targets the conjunctiva. In some embodiments, the double-stranded RNA molecule targets the cornea.

The conjunctiva comprises the bulbar conjunctiva, the palpebral conjunctiva, the ocular conjunctiva and the fornix conjunctiva. In some embodiments, the double-stranded RNA molecule is for administration to the bulbar conjunctiva. In some embodiments, the double-stranded RNA molecule is for administration to the palpebral conjunctiva. In some embodiments, the double-stranded RNA molecule is for administration to the ocular conjunctiva. In some embodiments, the double-stranded RNA molecule is for administration to the fornix conjunctiva. In some embodiments the double-stranded RNA molecule is for administration to one or more of the bulbar conjunctiva, the palpebral conjunctiva, the ocular conjunctiva and the fornix conjunctiva. In some embodiments, the double-stranded RNA molecule targets the bulbar conjunctiva. In some embodiments, the double-stranded RNA molecule targets the palpebral conjunctiva. In some embodiments, the double-stranded RNA molecule targets the ocular conjunctiva. In some embodiments, the double-stranded RNA molecule targets the fornix conjunctiva. In some embodiments the double-stranded RNA molecule targets one or more of the bulbar conjunctiva, the palpebral conjunctiva, the ocular conjunctiva and the fornix conjunctiva.

In some embodiments the double-stranded RNA molecule may be for administration to the cornea and the conjunctiva. In some embodiments the double-stranded RNA molecule may target the cornea and the conjunctiva.

In some embodiments, the double-stranded RNA molecule of the invention may be administered in combination with albumin. The albumin may be serum albumin, such as mouse serum albumin or human serum albumin.

### Complementarity to the Target Sequence

The contiguous nucleotide sequence of the double-stranded RNA molecule of the invention may be complementary to a target nucleic acid sequence, for example the nucleic acid sequence of a target mRNA.

In some embodiments, the contiguous nucleotide sequence may be fully, such as about 100%, complementary to a target nucleic acid sequence.

In some embodiments, the contiguous nucleotide sequence may be partially complementary to a target nucleic acid sequence, for example at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% complementary to the target nucleic acid sequence.

In some embodiments, the contiguous nucleotide sequences may include 1, 2, 3, 4, 5, 6, 7, 8 or more mismatches, wherein a mismatch is a nucleotide within the contiguous nucleotide sequence which does not base pair with its target.

The target nucleic acid sequence may be a target nucleic acid sequence, such as an mRNA sequence, expressed or present in the eye.

In some embodiments, the target may be any target present in the eye. In some embodiments, the target nucleic acid sequence may be any nucleic acid sequence expressed or present in the eye. In some embodiments, the target nucleic acid sequence may be any mRNA sequence expressed or present in the eye. It will be understood that reference herein to the eye includes any part of the eye. It will also be understood that reference herein to the eye includes a combination of any parts of the eye, including, but not limited to, the front of the eye or all parts of the eye.

In some embodiments, the target may be any target associated with an eye pathology, eye disease or eye condition. In some embodiments, the target may be any target associated with one or more eye pathologies, eye diseases or eye conditions.

In some embodiments, the target nucleic acid sequence may be any nucleic acid sequence associated with an eye pathology, eye disease or eye condition. In some embodiments, the target nucleic acid sequence may be any nucleic acid sequence associated with one or more eye pathologies, eye diseases or eye conditions.

In some embodiments, the target nucleic acid sequence may be any mRNA sequence associated with an eye pathology, eye disease or eye condition. In some embodiments, the target nucleic acid sequence may be any mRNA sequence associated with one or more eye pathologies, eye diseases or eye conditions.

It will be understood that a target (or target nucleic acid sequence or target mRNA sequence) associated with an eye pathology, eye disease or eye condition includes, but is not limited to, any target (or target nucleic acid sequence or target mRNA sequence) that causes an eye pathology, eye disease or eye condition, contributes to an eye pathology, eye disease or eye condition or is a marker of an eye pathology, eye disease or eye condition.

An example of a specific target that can be used in the present invention is AHA-1. It will be understood that AHA-1 is a non-limiting example of a target. It will also be understood that multiple other targets are encompassed by the present invention.

AHA1 (or AHA-1), in particular the AHA1 gene, is also known as p38; AHSA1; hAha1; C14orf3. In the context of the present invention, it will be understood that “AHA1”, “p38”, “AHSA1”, “hAha1” and C14orf3 are interchangeable terms.

In some embodiments, the target is an AHA-1 nucleic acid sequence.

The AHA-1 target nucleic acid sequence may comprise or consist of SEQ ID NO: 1 or SEQ ID NO: 2.

SEQ ID NO: 1 (human AHA1 target sequence:
pos. 488-507, NCBI Reference Sequence:
NM_012111.3):
5′-AATCTCGTGGCCTTAATGAAA-3′
SEQ ID NO: 2 (Rabbit AHA1 target sequence:
pos. 548-567, NCBI Reference Sequence:
XM_002719625.3):
5′-AATCTCGTGGCCTTAATGAAG-3

It will be understood that a “wobble” base pair mismatch is present in SEQ ID NO: 2 compared to SEQ ID NO: 1 (i.e. the A to G in the last nucleotide position is different between SEQ ID NO: 1 and SEQ ID NO: 2). Without wishing to be bound by theory, this mismatch should not affect the activity of the double-stranded RNA as either A or G can still hybridise to the complementary U in the contiguous nucleotide sequence of the strand of the double-stranded RNA molecule (e.g. by wobble base pairing). If a wobble base pair mismatch is present, it will be understood that this mismatch should not affect the activity of the double-stranded RNA molecule.

In some embodiments, the contiguous nucleotide sequence comprises or consists of a nucleotide sequence that is complementary to an AHA-1 target sequence.

In some embodiments, the contiguous nucleotide sequence comprises or consists of a nucleotide sequence that is complementary to SEQ ID NO: 1.

In some embodiments, the contiguous nucleotide sequence comprises or consists of a nucleotide sequence that is complementary to SEQ ID NO: 2.

In some embodiments, the contiguous nucleotide sequence comprises or consists of SEQ ID NO: 3.

SEQ ID NO: 3 (AHA-1 siRNA antisense strand):
5′-UUUCAUUAAGGCCACGAGAUU-3′

In some embodiments, AHA-1 may be expressed in the bulbar conjunctiva and/or the palpebral conjunctiva. In some embodiments, a reduction in expression of AHA-1 may be observed in the bulbar conjunctiva and/or the palpebral conjunctiva.

### Inhibition of the Target

In some embodiments, the double-stranded RNA molecule of the present invention may be capable of inhibiting the expression of a target. In other words, the double-stranded RNA molecule may be capable of decreasing the level of expression of a target.

The target may be a target nucleic acid sequence (such as a target mRNA sequence) or a target protein.

In some embodiments, the double-stranded RNA molecule of the present invention may be capable of inhibiting the expression of a target by at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%, relative to a control.

In some embodiments, the double-stranded RNA molecule of the present invention may be capable of inhibiting the expression of a target mRNA by at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100% relative to a control.

In some embodiments, the double-stranded RNA molecule of the present invention may be capable of inhibiting the expression of a target protein by at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100% relative to a control.

It will be understood that the % inhibition in the expression of a target referred to above will be a % decrease relative to a control, wherein the term “control” refers to expression of a target in a cell that has not been exposed to the double-stranded RNA molecule of the invention.

In some embodiments, the control may be a mock transfection, for example, treatment of cells with PBS. Herein, a decrease in expression of a target includes a decrease in the levels of a target mRNA and/or a decrease in the levels of a target protein.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides, comprise a ribose sugar moiety, a nucleobase moiety and one or more phosphate groups (which is/are absent in nucleosides). Nucleosides and nucleotides may also interchangeably be referred to as “units” or “monomers”.

### Modified Double-Stranded RNA Molecule

The double-stranded RNA molecule of the invention may be a modified double-stranded RNA molecule.

The term “modified double-stranded RNA molecule” encompasses a double-stranded RNA molecule comprising one or more sugar-modified nucleosides and/or modified internucleoside linkages.

In some embodiments, the double-stranded RNA molecule or contiguous nucleotide sequence thereof may include modified nucleobases which function as the shown nucleobase in base pairing, for example 5-methyl cytosine may be used in place of methyl cytosine. Inosine may be used as a universal base.

It is understood that the contiguous nucleobase sequences (motif sequence) can be modified to, for example, increase nuclease resistance and/or binding affinity to the target nucleic acid.

The pattern in which the modified nucleosides (such as high affinity modified nucleosides) are incorporated into the oligonucleotide sequence is generally termed oligonucleotide design.

In some embodiments, high affinity modified nucleosides may be used.

In an embodiment, the double-stranded RNA molecules comprise at least 1 modified nucleoside, such as at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49 or at least 50 modified nucleosides.

Suitable modifications are described herein.

### Modified Internucleoside Linkage

In some embodiments, the double-stranded RNA molecule of the invention may comprise one or more modified internucleoside linkage.

The term “modified internucleoside linkage” is defined as generally understood by the skilled person as linkages, other than phosphodiester (PO) linkages, that covalently couple two nucleosides together. The double-stranded RNA molecule of the invention may therefore comprise one or more modified internucleoside linkages such as one or more phosphorothioate internucleoside linkage.

In some embodiments at least 50% of the internucleoside linkages in the double-stranded RNA molecule, or contiguous nucleotide sequence thereof, are modified, such as at least 60%, such as at least 70%, such as at least 75%, such as at least 80%, such as at least 85%, such as at least 90%, such as at least 95% or more of the internucleoside linkages in the double-stranded RNA molecule, or contiguous nucleotide sequence thereof, are modified. In some embodiments all of the internucleoside linkages of the double-stranded RNA molecule, or contiguous nucleotide sequence thereof, are modified.

In some embodiments, one or more, or all, of the modified internucleoside linkages comprise a phosphorothioate linkage.

In some embodiments at least 50% of the internucleoside linkages in the double-stranded RNA molecule, or contiguous nucleotide sequence thereof, are phosphorothioate, such as at least 60%, such as at least 70%, such as at least 75%, such as at least 80%, such as at least 85%, such as at least 90%, such as at least 95% or more of the internucleoside linkages in the double-stranded RNA molecule, or contiguous nucleotide sequence thereof, are phosphorothioate. In some embodiments all of the internucleoside linkages of the double-stranded RNA molecule, or contiguous nucleotide sequence thereof, are phosphorothioate.

In a further embodiment, the double-stranded RNA molecule may comprise at least one modified internucleoside linkage. In some embodiments, at least 75%, such as all, of the internucleoside linkages within the contiguous nucleotide sequence are phosphorothioate or boranophosphate internucleoside linkages.

In some embodiments, all the internucleoside linkages of the contiguous nucleotide sequence of the double-stranded RNA molecule may be phosphorothioate, or all the internucleoside linkages of the double-stranded RNA molecule may be phosphorothioate linkages.

### Modified Nucleoside

In some embodiments, the double-stranded RNA molecule of the invention may comprise one or more modified nucleoside(s).

The term “modified nucleoside” or “nucleoside modification” as used herein refers to nucleosides modified as compared to the equivalent DNA or RNA nucleoside by the introduction of one or more modifications of the sugar moiety or the (nucleo) base moiety.

In some embodiments, one or more of the modified nucleosides of the double-stranded RNA molecules of the invention may comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow

Watson Crick base pairing. Exemplary modified nucleosides which may be used in the double-stranded RNA molecule of the invention include LNA, 2′-O-MOE, 2′oMe and morpholino nucleoside analogues.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization.

In the context of the present invention the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al., 2012, Accounts of Chemical Research, 45, 2055-2065 and Bergstrom, 2009, Curr. Protoc. Nucleic Acid Chem., 37, 1.4.1-1.4.32.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used. 5-methyl cytosine may be denoted as “E”.

### High Affinity Modified Nucleosides

A high affinity modified nucleoside is a modified nucleoside which, when incorporated into an oligonucleotide, enhances the affinity of the oligonucleotide for its complementary target, for example as measured by the melting temperature (T^{m}). A high affinity modified nucleoside of the present invention preferably results in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann, Nucl. Acid Res., 1997, 25, 4429-4443 and Uhlmann; Curr. Opinion in Drug Development, 2000, 3 (2), 203-213).

### Sugar Modifications

The double-stranded RNA molecules of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO 2011/017521) or tricyclic nucleic acids (WO 2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

2′ sugar modified nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA (2′oMe). 2′-alkoxy-RNA, 2′-O-methoxyethyl-RNA (MOE), 2′-amino-DNA, 2′-Fluoro-RNA, and 2′-F-ANA nucleoside. For further examples, see e.g. Freier & Altmann; Nucl. Acid Res., 1997, 25, 4429-4443 and Uhlmann; Curr. Opinion in Drug Development 2000, 3 (2), 203-213, and Deleavey and Damha, Chemistry and Biology 2012, 19, 937. Below are illustrations of some 2′ substituted modified nucleosides.

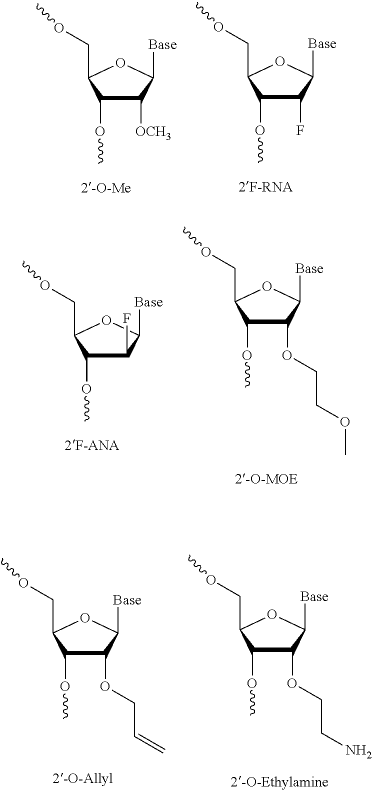

In relation to the present invention 2′ substituted sugar modified nucleosides do not include 2′ bridged nucleosides like LNA.

In some embodiments, the double-stranded RNA molecule comprises one or more sugar modified nucleosides, such as 2′ sugar modified nucleosides.

In some embodiments, the double-stranded RNA molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 sugar modified nucleosides.

In some embodiments, the double-stranded RNA molecule of the invention comprise one or more 2′ sugar modified nucleoside independently selected from the group consisting of 2′-O-alkyl-RNA, 2′-O-methyl-RNA (2′oMe), 2′-O-methoxyethyl-RNA (2′MOE), 2′-alkoxy-RNA, 2′-amino-DNA, 2′-fluro-RNA 2′-fluoro-DNA, arabino nucleic acid (ANA), 2′-fluoro-ANA and LNA nucleosides. In some embodiments, one or more of the modified nucleoside(s) may be a LNA. In some embodiments, the 2′ sugar modified nucleoside is an affinity enhancing 2′ sugar modified nucleoside.

Locked Nucleic Acid Nucleosides (LNA nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring.

These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett., 12, 73-76, Seth et al., J. Org. Chem., 2010, Vol 75 (5) pp. 1569-81, Mitsuoka et al., Nucleic Acids Research, 2009, 37 (4), 1225-1238, and Wan and Seth, J. Medical Chemistry, 2016, 59, 9645-9667.

Further non limiting, exemplary LNA nucleosides are disclosed in Scheme 1.

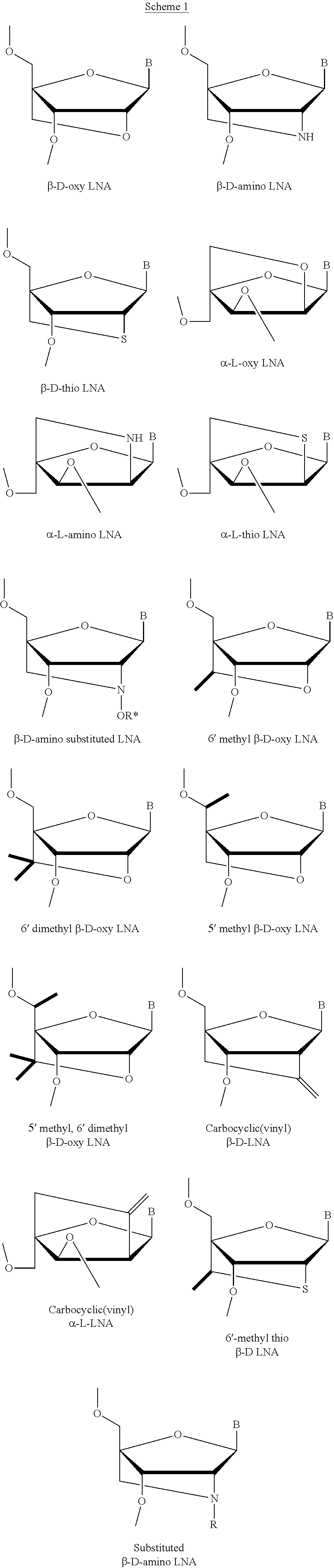

Particular LNA nucleosides are beta-D-oxy-LNA, 6′-methyl-beta-D-oxy LNA such as(S)-6′-methyl-beta-D-oxy-LNA (ScET) and ENA.

A particularly advantageous LNA is beta-D-oxy-LNA.

### Morpholino Oligonucleotides

In some embodiments, the double-stranded RNA molecule of the invention comprises or consists of morpholino nucleosides (i.e. is a Morpholino oligomer and as a phosphorodiamidate Morpholino oligomer (PMO)). Splice modulating morpholino oligonucleotides have been approved for clinical use—see for example eteplirsen, a 30nt morpholino oligonucleotide targeting a frame shift mutation in DMD, used to treat Duchenne muscular dystrophy. Morpholino oligonucleotides have nucleases attached to six membered morpholino rings rather ribose, such as methylenemorpholine rings linked through phosphorodiamidate groups, for example as illustrated by the following illustration of 4 consecutive morpholino nucleotides:

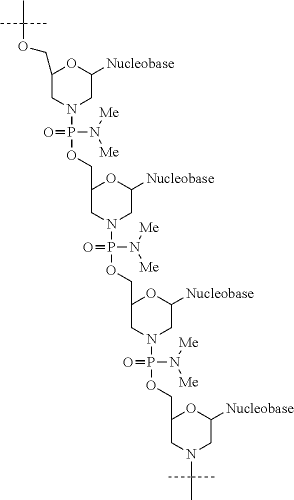

In some embodiments, double-stranded RNA molecules of the invention may be, for example 8-50 morpholino nucleotides in length.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U).

It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al., 2012, Accounts of Chemical Research, 45, 2055 and Bergstrom, 2009, Curr. Protoc. Nucleic Acid Chem., 37, 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence.

It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

### Identity

The term “identity” as used herein, refers to the proportion of nucleotides (expressed in percent) of a contiguous nucleotide sequence in a nucleic acid molecule (e.g. siRNA molecule) which across the contiguous nucleotide sequence, are identical to a reference sequence (e.g. a sequence motif).

The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches x 100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

It is therefore to be understood that there is a relationship between identity and complementarity such that contiguous nucleotide sequences within the double-stranded RNA molecule of the invention that are complementary to a target sequence also share a percentage of identity with said complementary sequence.

### Hybridization

The terms “hybridizing” or “hybridizes” as used herein are to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=-RTIn (K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95:1460-1465 using appropriately derived thermodynamic parameters described by Sugimoto et al., 1995, Biochemistry 34:11211-11216 and McTigue et al., 2004, Biochemistry 43:5388-5405.

In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal. In some embodiments the oligonucleotides hybridize to a target nucleic acid with an estimated ΔG° value of −10 to −60 kcal, such as −12 to −40, such as from −15 to −30 kcal, or −16 to −27 kcal such as −18 to −25 kcal.

### Salts

The term “salts” as used herein conforms to its generally known meaning, i.e. an ionic assembly of anions and cations.

The invention provides for pharmaceutically acceptable salts of the double-stranded RNA molecule of the invention. In other words, the invention provides for a double-stranded RNA molecule of the invention wherein the double-stranded RNA molecule is in the form of a pharmaceutically acceptable salt.

In some embodiments the pharmaceutically acceptable salt may be a sodium salt or a potassium salt.

The invention provides for a pharmaceutically acceptable sodium salt of the double-stranded RNA molecule of the invention.

The invention provides for a pharmaceutically acceptable potassium salt of the double-stranded RNA molecule of the invention.

### Delivery of Double-Stranded RNA Molecules

The double-stranded RNA molecules of the invention can be encapsulated in a lipid-based delivery vehicle, covalently linked to or encapsulated in a dendrimer, or conjugated to an aptamer.

This may be for the purpose of delivering the double-stranded RNA molecule of the invention to the targeted cells and/or to improve the pharmacokinetics of the double-stranded RNA molecule.

Examples of lipid-based delivery vehicles include oil-in-water emulsions, micelles, liposomes, and lipid nanoparticles.

It will be understood that, as the double-stranded RNA molecule of the invention is conjugated to at least one conjugate moiety, encapsulation of the double-stranded RNA molecule is in addition to the conjugation of the double-stranded RNA molecule to the at least one conjugate moiety, which may provide additional advantages compared to conjugation to a conjugate moiety alone.

In some embodiments, the double-stranded RNA molecule of the invention may be administered in combination with albumin. The albumin may be serum albumin, such as mouse serum albumin or human serum albumin. Without wishing to be bound by theory, the albumin may function as a transport vehicle and/or may facilitate or enhance uptake of the double-stranded RNA molecule of the invention into a relevant tissue or cell.

### Pharmaceutical Compositions

The invention provides for a pharmaceutical composition comprising the double-stranded RNA molecule of the invention, and a pharmaceutically acceptable diluent, solvent, carrier, salt and/or adjuvant.

The invention provides for a pharmaceutical composition comprising the double-stranded RNA molecule of the invention, and a pharmaceutically acceptable salt. For example, the salt may comprise a metal cation, such as a sodium salt or a potassium salt.

The invention provides for a pharmaceutical composition of the invention, wherein the pharmaceutical composition comprises the double-stranded RNA molecule of the invention, and an aqueous diluent or solvent.

The invention provides for a solution, such as a phosphate buffered saline solution of the double-stranded RNA molecule of the invention. In some embodiments, the solution, such as phosphate buffered saline solution, of the invention is a sterile solution.

The pharmaceutical composition of the invention can be topically administered to the eye (e.g. ophthalmic administration). It is to be understood that the double-stranded RNA molecule of the invention can be administered to any part of the eye.

In some embodiments, the pharmaceutical composition is for administration to the front of the eye. In some embodiments, the pharmaceutical composition is for administration to the conjunctiva. In some embodiments, the pharmaceutical composition is for administration to the cornea. In some embodiments, the pharmaceutical composition is for administration to the bulbar conjunctiva. In some embodiments, the pharmaceutical composition is for administration to the palpebral conjunctiva. In some embodiments, the pharmaceutical composition is for administration to the ocular conjunctiva. In some embodiments, the pharmaceutical composition is for administration to the fornix conjunctiva. In some embodiments, the pharmaceutical composition is for administration to one or more of the bulbar conjunctiva, the palpebral conjunctiva, the ocular conjunctiva, and the fornix conjunctiva.

In some embodiments, the pharmaceutical composition of the invention may comprise albumin. The albumin may be serum albumin, such as mouse serum albumin or human serum albumin.

### Method of Manufacture

In a further aspect, the invention provides methods for manufacturing the double-stranded RNA molecule of the invention comprising reacting nucleotide units and thereby forming covalently linked contiguous nucleotide units comprised in the oligonucleotide. The method may use phophoramidite chemistry (see for example Caruthers et al, 1987, Methods in

Enzymology vol. 154, pages 287-313). In a further embodiment the method further comprises reacting the contiguous nucleotide sequence with a conjugating moiety to attach (e.g. covalently attach) the conjugate moiety to the double-stranded RNA molecule. In a further aspect a method is provided for manufacturing the composition of the invention, comprising mixing the double-stranded RNA molecule of the invention with a pharmaceutically acceptable diluent, solvent, carrier, salt and/or adjuvant.

### Treatment

The term “treatment” as used herein refers to both treatment of an existing disease (e.g. a disease or disorder as herein referred to), or prevention of a disease, i.e. prophylaxis. It will therefore be recognized that treatment, as referred to herein may in some embodiments be prophylactic.

The invention provides for a method for treating or preventing a disease, comprising administering a therapeutically or prophylactically effective amount of a double-stranded RNA molecule of the invention or a pharmaceutical composition of the invention to a subject suffering from or susceptible to a disease.

The invention provides for a double-stranded RNA molecule of the invention for use as a medicament for the treatment of a disease.

The invention provides for a double-stranded RNA molecule of the invention for use in therapy.

The invention provides for a double-stranded RNA molecule of the invention for the preparation of a medicament for treatment or prevention of a disease.

The invention provides for a pharmaceutical composition of the invention for use as a medicament.

The invention provides for a pharmaceutical composition of the invention for use in therapy.

The invention provides for a pharmaceutical composition of the invention for the preparation of a medicament for treatment or prevention of a disease.

The disease may be a disease associated with the eye. The disease may be a disease that affects the eye.

The disease may be an infection of (or associated with) the eye. The disease may be a type of inflammation of (or associated with) the eye.

In some embodiments, the disease may be conjunctivitis, dry eyes or inflammation of the eye.

In some embodiments, the subject to be treated is an animal, preferably a mammal such as a mouse, rat, hamster, or monkey, or preferably a human. In some embodiments, the subject is a human.

In some embodiments, the double-stranded RNA molecule of the invention or pharmaceutical composition of the invention is for use in a combination treatment with another therapeutic agent.

### Administration Strategy

It will be understood that the double-stranded RNA molecule of the invention or pharmaceutical composition of the invention can be administered to a subject once, or can be administered over a period of hours, days, weeks, months or years.

In some embodiments, the administration of the double-stranded RNA molecule or pharmaceutical composition may be chronic administration.

In some embodiments, the double-stranded RNA molecule or pharmaceutical composition of the invention is administered to only one eye.

In some embodiments, the double-stranded RNA molecule or pharmaceutical composition of the invention is administered to both eyes. The administration to both eyes can be simultaneously or sequentially.

In some embodiments, the double-stranded RNA molecule is administered to the (or each) eye once per day, twice per day, three times per day or more than three times per day. For example, the double-stranded RNA molecule may be administered to the (or each) eye 1, 2, 3, 4, 5, 6, 7, 8, 9, 10 or more times per day.

In some embodiments, the double-stranded RNA molecule may be administered to the (or each) eye every 1 hour, every 2 hours, every 3 hours, every 4 hours, every 5 hours, every 6 hours, every 7 hours, every 8 hours, every 9 hours, every 10 hours, every 11 hours or every 12 hours. In some embodiments, the double-stranded RNA molecule may be administered to the (or each) eye every 4 hours.

In some embodiments, the double-stranded RNA molecule is administered to the (or each) eye for less than one day, or for one day, or two consecutive days, three consecutive days, four consecutive days, five consecutive days, six consecutive days, seven consecutive days, eight consecutive days, nine consecutive days, ten consecutive days or more than ten consecutive days. For example, the double-stranded RNA molecule may be administered to the (or each) eye for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more than 31 day(s).

In some embodiments, the double-stranded RNA molecule is administered to the (or each) eye for a period of one week, two weeks, three weeks, four weeks, five weeks, six weeks or more than six weeks. For example, the double-stranded RNA molecule may be administered to the (or each) eye for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or more than 52 week(s).

In some embodiments, the double-stranded RNA molecule is administered to the (or each) eye for a period of one month, two months, three months, four months, five months, six months or more than six months. For example, the double-stranded RNA molecule may be administered to the (or each) eye for 1, 2, 3, 4, 5, 6, 7, 8, 9, 10, 11, 12 or more than 12 month(s).

In some embodiments, the double-stranded RNA molecule is administered to the (or each) eye for a period of one year, two years, three years, four years, five years, or more than five months. For example, the double-stranded RNA molecule may be administered to the (or each) eye for 1, 2, 3, 4, 5, 6, 7, 8, 9, 10, 11, 12, 13, 14, 15, 16, 17, 18, 19, 20, 21, 22, 23, 24, 25 or more than 25 year(s).

### Method for Modulating Expression

The invention provides for a method for modulating the expression of a target in a cell, said method comprising administering a double-stranded RNA molecule of the invention, or the pharmaceutical composition of the invention, in an effective amount to said cell.

In some embodiments the method is an in vitro method.

In some embodiments the method is an in vivo method.

In some embodiments, the cell is an animal cell, preferably a mammalian cell such as a mouse cell, rat cell, hamster cell, or monkey cell, or preferably a human cell.

In some embodiments, the cell is a mammalian cell.

In some embodiments, the cell is a human cell.

In some embodiments, the cell is a cell of the eye. In some embodiments, the cell is a cell of the human eye.

In some embodiments, the cell may be collected by a sampling medical device. In some embodiments, the cell may be from a collection of conjunctival cells from the ocular surface of the eye.

### Applications

The double-stranded RNA molecule of the invention may be utilized as research reagents for, for example, diagnostics, therapeutics and prophylaxis.

In research, such double-stranded RNA molecules may be used to inhibit the expression of a target expressed in the eye in experimental animals thereby facilitating functional analysis of the target or an appraisal of its usefulness as a target for therapeutic intervention.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

New Zealand white rabbits were dosed by topical administration in both left and right eyes, 3 times a day with a least 4 h between dosing for 5 days with 20 μl of a 25 μg/μL solution (500 μg pr dose) of either naked or C16-, C22-, or cholesterol-conjugated siRNA molecules comprising the sequence of SEQ ID NO: 3 formulated in PBS. Four days after last dosing, rabbits were sacrificed and palpebral conjunctiva samples were taken from each eye (Saline n=4, siRNA treated groups n=6) and analysed for AHSA1 mRNA knockdown by digital droplet PCR.

The conjunctiva tissue was homogenized using the TissueLyser II (Qiagen) in 500 μL MagnaPure Tissue Lysis buffer (Roche LifeScience) after adding a metal bead and mRNA was extracted from 350 μL Lysis buffer using the MagNA Pure 96 system according to manufactorer's instructions (Roche LifeScience) and extracted in 50 μL RNAse free water. cDNA synthesis was performed with 4 μL input RNA using IScript Advanced cDNA Synthesis Kit for RT-qPCR (Bio-Rad) and 2 μL was used as input for digital droplet PCR using ddPCR supermix for probes (no dUTP) (Bio-Rad) according to Manufactor's protocol.

The following TaqMan gene expression assays were used:

AHSA1 (FAM): Oc06762465_g1 (Catalog number: 4351372, TaqMan Thermofisher Scientific) and HPRT1 (VIC): Oc03399461_m1 (Catalog number: 4331182, TaqMan Thermofisher Scientific)

AHSA1 mRNA concentrations were quantified relative to the housekeeping gene HPRT using QuantaSoft Software (Bio-Rad) and normalized to PBS treated rabbits (set to value of 1).

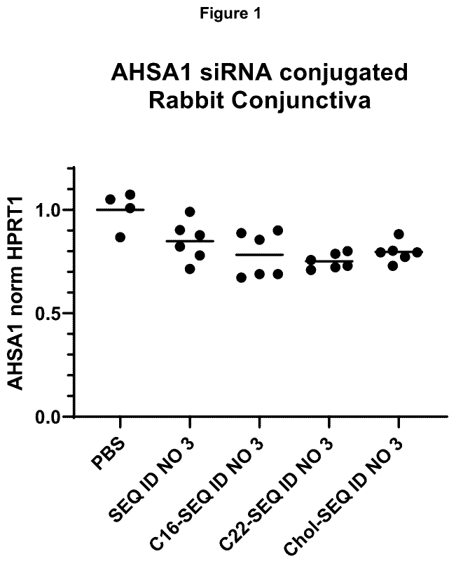

The results for are shown in FIG. 1. 20-25% knockdown in the entire conjunctiva was observed for naked siRNA molecules comprising the sequence of SEQ ID NO: 3, C16-conjugated siRNA molecules comprising the sequence of SEQ ID NO: 3, C22-conjugated siRNA molecules comprising the sequence of SEQ ID NO: 3 and cholesterol-conjugated siRNA molecules comprising the sequence of SEQ ID NO: 3, indicating superior knockdown with C16, C22 and Cholesterol AHSA1 siRNA compared to naked AHSA1 siRNA.

### Example 2

New Zealand white rabbits were dosed by topical administration in the eye, 3 times pr day with a least 4 h between for 5 days with 20 μl of a 25 μg/μL solution (500 μg pr dose) of either naked or C16-, C22-, or cholesterol-conjugated siRNA molecules comprising the sequence of SEQ ID NO: 3 in PBS. Four days after last dosing, rabbits were sacrificed and palpebral conjunctiva samples were taken (Saline n=4, siRNA treated groups n=6) and analysed for AHSA1 siRNA content analysis by hELISA.

The conjunctiva tissue (˜5 mg) was homogenized using the TissueLyser II (Qiagen) in 500 μL MagnaPure Tissue Lysis buffer (Roche LifeScience) after adding a metal bead. siRNA content was determined using hELISA, using a biotinylated capture probe and a digoxigenin-conjugated detection probe binding to the antisense siRNA strand. The resulted lysates were diluted and incubated with 35 nM biotinylated capture probe and 30 nM digoxigenin-coupled detection probe for 30 min at room temperature in SSCT buffer (5+ saline sodium citrate buffer [SSC Buffer 20×Concentrate, Sigma-Aldrich, no. 6639] containing 0.05% Tween 20 [Sigma-Aldrich, no. P9416]) in a 96-well plate. The assembled complex is then captured on a streptavidine-coated ELISA plate (Nunc 436014) for 1 h, and after three washing steps with 2×SSCT buffer, each well is incubated with an anti-digoxigenin-alkaline phosphatase (AP)-Fab fragment (Roche, no. 11093274910) for 1 h at room temperature. After three additional washing steps, BluePhos substrate (Kirkegaard & Perry Labs [KPL], no. 50-88-00) was added to the plates, and color development was measured spectrophotometrically at 615 nm after 20 min. For the oligo content analysis, several dilutions of each sample (50×, 100×, 200×, 400×, 800×, and 1,600×) are measured. Calculating back to the respective tissue weight and making an average of the valid values from different dilutions (i.e., the readouts within the linear range of the standard curve) generate the drug concentration in nmol/g conjunctiva tissue weight.

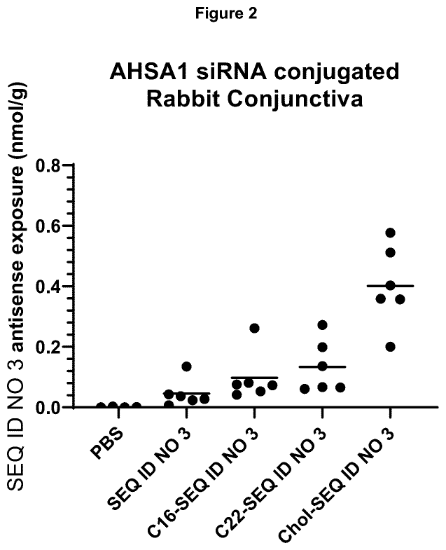

The results for are show in FIG. 2. Increased content in the conjunctiva was observed for naked siRNA molecules comprising the sequence of SEQ ID NO: 3, C16-conjugated siRNA molecules comprising the sequence of SEQ ID NO: 3, C22-conjugated siRNA molecules comprising the sequence of SEQ ID NO: 3 and cholesterol-conjugated siRNA molecules comprising the sequence of SEQ ID NO: 3, indicating superior content with C16, C22 and especially with Cholesterol conjugated AHA1 siRNA compared to naked siRNA.

### Example 3

New Zealand white rabbits were dosed by topical administration in the eye, 3 times pr day with a least 4 h between for 5 days with 20 μl of a 25 μg/μL solution (500 μg pr dose) of either naked or C16-, C22-, or cholesterol-conjugated siRNA molecules comprising the sequence of SEQ ID NO: 3 in PBS. Four days after last dosing EYEPRIM (OPIA technologies) samples were taken from the bulbar conjunctiva (Saline n=4, siRNA treated groups n=6). Following euthanasia of the animal, bulbar conjunctiva will be exposed and an EYEPRIM membrane will be pressed against the inferior bulbar conjunctiva for 3 seconds. Then the membrane was removed from the EYEPRIM. Doing so, the membrane was held with a forcept at the time of ejection to avoid that the membrane falls/flies away. The membrane was snap frozen into a 2 mL Eppendorf tube.

The EYEPRIM samples were homogenized using the TissueLyser II (Qiagen) in 500 μL MagnaPure Tissue Lysis buffer (Roche LifeScience) after adding a metal bead and mRNA was extracted from 350 μL Lysis buffer using the MagNA Pure 96 system according to manufactorer's instructions (Roche LifeScience) and extracted in 50 L RNAse free water. cDNA synthesis was performed with 4 μL input RNA using IScript Advanced cDNA Synthesis Kit for RT-qPCR (Bio-Rad) and 2 μL was used as input for digital droplet PCR using ddPCR supermix for probes (no dUTP) (Bio-Rad) according to Manufactor's protocol.

The following TaqMan gene expression assays were used:

AHSA1 (FAM): Oc06762465_g1 (Catalog number: 4351372, TaqMan Thermofisher Scientific) and HPRT1 (VIC): Oc03399461_m1 (Catalog number: 4331182. TaqMan Thermolisher Scientific).

AHSA1 mRNA concentrations were quantified relative to the housekeeping gene HPRT using QuantaSoft Software (Bio-Rad) and normalized to PBS treated rabbits (PBS set to 1)

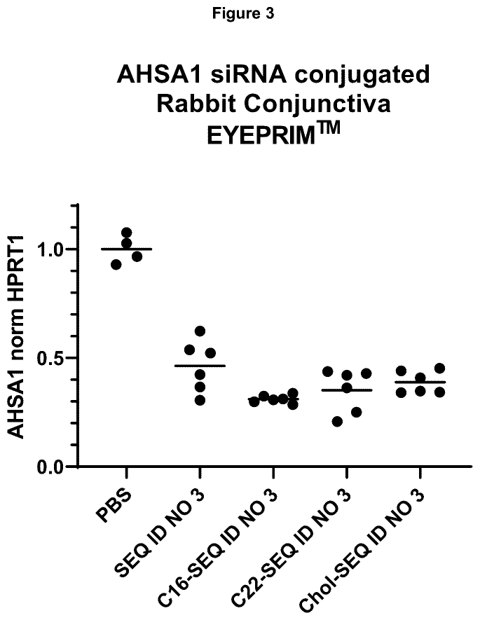

The results are show in FIG. 3. 69% knockdown (C16-conjugated SEQ ID NO: 3), 65% knockdown (C22-conjugated SEQ ID NO: 3) and 61% knockdown (Cholesterol-conjugated SEQ ID NO: 3) in the conjunctiva EYEPRIM samples were compared to 54% for naked siRNA (SEQ ID NO: 3), significant superior knockdown with C16 (p=0.02 Student's t-test), and superior knockdown with C22 and Cholesterol conjugated AHSA1 siRNA compared to naked siRNA.

### Example 4

### Isothermal Titration Calorimetry (ITC)

Experiments were conducted in an auto-PEAQ ITC (Malvern Panalytical, Malvern, United Kingdom) in the assay setup where the target protein is present in the syringe, and was injected into FA-siRNA samples placed in the cell. The concentration of mouse serum albumin (MSA, cat. No. A3139-100 mg, essentially fatty acid-free) (25-450 μM) in the syringe and FA-siRNA (3-60 μM) in the cell, were adjusted using the filtered and degassed buffer from the dialysis, based on their measured stoichiometry and affinity of binding. ITC analyses were run at 25° C. with the following experimental parameters: stirrer speed: 750 rpm, spacing: 150 s, injection duration: 3 s, reference power 10 ucal/s. The initial delay was set to 60 s and the filter period to 5 s with 13 or 19 injections with the titration volume 2.0 or 3.0, respectively, performed to collect enthalpy data for the analysed interaction. Control titrations to correct for heat of dilution were performed (buffer into FA-siRNA, MSA into buffer, and buffer into buffer). MSA was dissolved in DPBS (cat. No. 14190-094, Gibco/Thermo Fisher Scientific, Basel, Switzerland) and further dialyzed using a DPBS with Slide-A-Lyzer Dialysis cassettes 20K MWCO (cat. No. 66003, Thermo Scientific™, Waltham, MA) prior to ITC analysis. FA-siRNA powders were solubilized in DPBS. The dialyzed protein and FA-siRNA concentrations were determined using UV spectroscopy, respectively, at 280 and 260 nm (according to the Beer-Lambert law equation). Offset and MSA heat of dilution were subtracted from the heat of reaction generated during the titration of FA-siRNA. The baseline was adjusted manually (if needed) and the corrected heat of reaction was fitted using a single site binding model in the MicroCal PEAQ ITC analysis software, version 1.3 (Malvern instrument, Malvern, United Kingdom). Control ITC experiment of MSA titration into warfarin (DPBS+2% DMSO (v/v)) was performed prior to the FA-siRNA ITC study.

### Analytical Ultracentrifugation (AUC)

Samples were analysed in sedimentation velocity (SV) and/or sedimentation equilibrium (SE) experiments on the analytical ultracentrifuge XLI and Optima-AUC (Beckman Coulter, CA) with absorbance detection (range of 260-295 nm) depending on the tested concentration (1-75 μM). The individual samples were dissolved in DPBS (cat. No. 14190-094, Gibco/Thermo Fisher Scientific, Basel, Switzerland) and diluted in DPB. AUC-SV experiments were run at 60,000 rpm and 20° C. using 3 and 12 mm Ti centerpieces (Nanolytics Instruments, Potsdam,

Germany) and an An-60 Ti rotor (Beckman Coulter, CA). SE experiments were performed in a multi-speed mode (rotor speed of 10, 15, and 20 krpm or 7, 10, and 14 krpm for FA Bis C16 and FA C24 conjugates, respectively) and the FA-siRNA conjugate concentration was set to 6 and 25 μM. 3 mm Ti centerpieces (Nanolytics Instruments, Potsdam, Germany) and an An-60 Ti rotor (Beckman Coulter, CA) were used in SE analysis. Buffer density and viscosity were determined experimentally using a densitometer and viscometer (DMA 5000 M and AMVn, respectively, Anton Paar, Aarau, Switzerland). The interactions of FA-siRNA conjugates and MSA were analysed in SV mode with absorbance (290-305 nm) and/or fluorescence detection using a fluorescence detection system (Aviv Biomedical, NJ).

### Results

The results are shown in FIG. 4, which provides biophysical analysis of different fatty acid conjugated AHA-1 specific siRNAs (FA-siRNA). Column 3: the propensity of the different fatty acid conjugates to exist in different oligomeric states at 25 μM concentration (Final Oligomeric State as measured by AUC); Column 4: monomer percentage of different FA-siRNAs when dissolved in 25 μM PBS; Column 5: binding affinity to mouse serum albumin (MSA; determined by ITC); Column 6: number of FA-siRNA conjugates bound to MSA.

The results demonstrate that (i) the binding strength of the fatty acid conjugated siRNA molecule can be tuned/modified by the length of the fatty acid; (ii) introduction of a double bond (e.g. stearic acid versus oleic acid) does not impact the binding of the fatty acid to albumin; and (iii) albumin binding can be exploited as a transport vehicle for the fatty acid conjugated siRNA molecule, and the binding strength adjusted as required.

### Example 5

New Zealand white rabbits were dosed by topical administration in both left and right eyes, 3 times a day with a least 4 h between dosing for 5 days with 20 μl of a 25 μg/μL solution (500 μg pr dose) of either naked or C16-, C22-, or cholesterol-conjugated siRNA molecules comprising the sequence of SEQ ID NO: 3 formulated in PBS. Three days after last dosing, rabbits were sacrificed and superior and inferior palpebral conjunctiva samples were taken from each eye (n=2 eyes) and tissue sections (n=3-8) were analysed for siRNA molecules distribution in the conjunctival tissue using in situ hybridization (ISH).

The conjunctival tissue was dissected and fixed in 10% NBF overnight followed by a standard dehydration process and embedded in paraffin the next day. Transversal conjunctival tissue sections (5 μm) were stained using the automated Ventana Discovery ULTRA autostainer (Roche Diagnostics). Automated protocols were designed for baking (60° C. for 20 min), deparaffinization (69° C. for 24 min) and ISH protease 3 treatment (one drop, 32 min). Slides were treated with DISC inhibitor (one drop, 12 min) and washed with reaction buffer (1×). The DIG-labeled probe targeting SEQ ID NO: 3 was diluted in miRCURY LNA miRNA ISH Buffer (1×in RNase-free water) to a final concentration of 0.25 nM, manually applied to the slides. Slides were denatured at 90° C. for 8 min before hybridization at 65° for 16 min. Slides then were washed with 0.1×SSC (5×at 54° C. for 4 min). DIG molecule was detected using anti-DIG-HRP (one drop, 4° C. with no heat) followed by applying DISC AMP TSA BF and DISC AMP H2O2 BF (one drop each, for 4 min without heat) and by DISC anti-BF HRP (one drop each, for 4 min without heat). HRP was detected with DAB (Brown) detection kit. Slides were counterstained with hematoxylin II (4 min without heat) followed by treatment with bluing reagent (4 min without heat) before mounting in EcoMount media. Slides were scanned using an Olympus VS120 slide scanner at 20× magnification to visualize the results. Quantification of positive staining was performed using Halo, Indica Labs (V3.6.4134.166).

The following reagents were used: ISH-Protease 3 (05273331001), Disc. Inhibitor (07017944001), Anti-DIG HRP (07256299001), Disc. Anti-BF HRP (07529422001)), DISC AMP TSA BF (07529422001), Hematoxylin II (05277965001), Bluing Reagent (05266769001), SSC Solution (10×) (05353947001), Reaction Buffer Concentrate (10×) (05353955001), DISC. ChromoMap DAB RUO (05266645001) all purchased from Roche Diagnostics. miRCURY LNA miRNA ISH Buffer (#1108512) and DIG-labeled probe targeting SEQ ID NO: 3 (Custom miRCURY LNA/5DiGN/CGTGGCCTTAATGAA/3DIG_N/) were purchased from Qiagen.

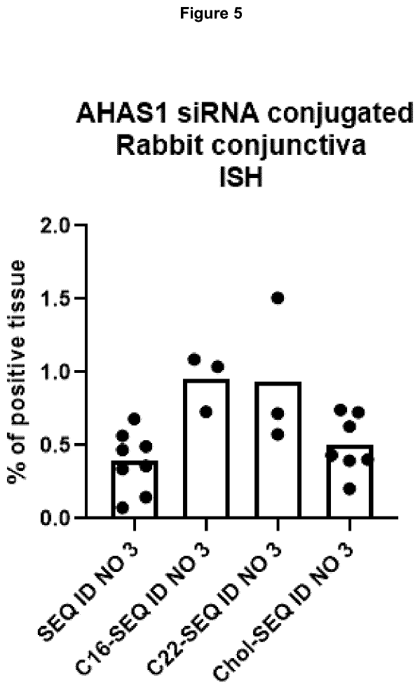

The results are shown in FIG. 5: 0.3-0.4% of conjunctival tissue ISH staining was observed for naked siRNA molecules comprising the sequence of SEQ ID NO:3, C16-conjugated siRNA molecules comprising the sequence of SEQ ID NO: 3, C22-conjugated siRNA molecules comprising the sequence of SEQ ID NO: 3 and cholesterol-conjugated siRNA molecules comprising the sequence of SEQ ID NO: 3, indicating superior tissue staining with C16, C22 and Cholesterol AHSA1 siRNA compared to naked AHSA1 siRNA, whereas C16 and C22 AHAS1 siRNA showed the most superior result compared to naked and Cholesterol AHAS1 siRNA. In addition, staining for naked AHSA1 siRNA was mainly located at the superficial conjunctiva whereas staining for C16, C22 and Cholesterol AHSA1 siRNA was also located in the stroma of the conjunctival tissue.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A double-stranded ribonucleic acid (RNA) molecule for topical administration to the eye, wherein the double-stranded RNA molecule is capable of binding to a target sequence, wherein the double-stranded RNA molecule comprises a first strand having a 5′ end and a 3′ end, and a second strand having a 5′ end and a 3′ end, wherein the first strand is complementary to the second strand, wherein the first strand comprises a contiguous nucleotide sequence of at least 8 nucleotides in length which is complementary to a target sequence, and wherein the double-stranded RNA molecule is conjugated to at least one conjugate moiety.

2. The double-stranded RNA molecule of claim 1, wherein:
(i) the double-stranded RNA molecule is:
(a) a siRNA molecule
(b) is capable of inhibiting the expression of a target;
(c) comprises one or more modified nucleoside(s); and/or
(d) is in the form of a pharmaceutically acceptable salt:
(ii) the conjugate moiety is:
(a) a fatty acid molecule or a cholesterol molecule; and/or
(b) is positioned at the 5′ end or the 3′ end of the first strand; or positioned at the 5′ end or the 3′ end of the second strand;
(iii) a linker is positioned between the double-stranded RNA molecule and the conjugate moiety; and/or
(iv) the contiguous nucleotide sequence:
(a) is at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length; or
(b) is at least 75%, at least 80%, at least 85%, at least 90%, at least 95% or 100% complementary to the target sequence;
(c) comprises 1, 2, 3, 4, 5, 6, 7, 8, or more mismatches to the target sequence;
(d) is complementary to an AHA-1 target sequence;
(e) comprises a nucleotide sequence that is complementary to SEQ ID NO:1 or SEQ ID NO: 2; and/or
(f) comprises SEQ ID NO: 3.

3-4. (canceled)

5. The double-stranded RNA molecule of claim 2, wherein:
(i) the fatty acid molecule:
(a) is selected from the list consisting of: C3, C4, C5, C6, C7, C8, C9, C10, C11, C12, C13, C14, C15, C16, C17, C18, C19, C20, C21, C22, C23, C24, C25, C26, C27, C28, C29, C30, C31, C32, C33, C34, C35, C36, C37, C38, C39, or C40;
(b) is branched or unbranched; and/or
(c) is saturated or unsaturated;
(ii) the cholesterol molecule is 3′-cholesteryl-TEG CPG, 5′-cholesterol-TEG-CE phosphoramidite, 5′-cholesterol-CE phosphoramidite, or cholesteryl-TEG-CE phosphoramidite;
(iii) the conjugate moiety is positioned at the 3′ end of the first strand;
(iv) the contiguous nucleic acid sequence:
(a) is 20, 21, 22, 23, or 24 nucleotides in length; and/or
(b) consists of SEQ ID NO: 3;
(v) the linker is C6, TEG, or a dinucleotide; and/or
(vi) the pharmaceutically acceptable salt is a sodium salt or a potassium salt.

6-11. (canceled)

12. The double-stranded RNA molecule of claim 5. [11,] wherein the fatty acid molecule:
(i) comprises one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more carbon double bonds;
(ii) comprises one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, or nineteen or more carbon triple bonds; and/or
(iii) is C3:0; C4:0; C4:1; C5:0; C5:1; C6:0; C6:1; C6:2; C7:0; C7:1; C7:2; C8:0; C8:1; C8:2; C8:3; C9:0; C9:1; C9:2; C9:3; C10:0; C10:1; C10:2; C10:3; C10:4; C11:0; C11:1; C11:2; C11:3; C11:4; C12:0; C12:1; C12:2; C12:3; C12:4; C12:5; C13:0; C13:1; C13:2; C13:3; C13:4; C13:5; C14:0; C14:1; C14:2; C14:3; C14:4; C14:5; C14:6; C15:0; C15:1; C15:2; C15:3; C15:4; C15:5; C15:6; C16:0; C16:1; C16:2; C16:3; C16:4; C16:5; C16:6; C16:7; C17:0; C17:1; C17:2; C17:3; C17:4; C17:5; C17:6; C17:7; C18:0; C18:1; C18:2; C18:3; C18:4; C18:5; C18:6; C18:7; C18:8; C19:0; C19:1; C19:2; C19:3; C19:4; C19:5; C19:6; C19:7; C19:8; C20:0; C20:1; C20:2; C20:3; C20:4; C20:5; C20:6; C20:7; C20:8; C20:9; C21:0; C21:1; C21:2; C21:3; C21:4; C21:5; C21:6; C21:7; C21:8; C21:9; C22:0; C22:1; C22:2; C22:3; C22:4; C22:5; C22:6; C22:7; C22:8; C22:9; C22:10; C23:0; C23:1; C23:2; C23:3; C23:4; C23:5; C23:6; C23:7; C23:8; C23:9; C23:10; C24:0; C24:1; C24:2; C24:3; C24:4; C24:5; C24:6; C24:7; C24:8; C24:9; C24:10; C24:11; C25:0; C25:1; C25:2; C25:3; C25:4; C25:5; C25:6; C25:7; C25:8; C25:9; C25:10; C25:11; C26:0; C26:1; C26:2; C26:3; C26:4; C26:5; C26:6; C26:7; C26:8; C26:9; C26:10; C26:11; C26:12; C27:0; C27:1; C27:2; C27:3; C27:4; C27:5; C27:6; C27:7; C27:8; C27:9; C27:10; C27:11; C27:12; C28:0; C28:1; C28:2; C28:3; C28:4; C28:5; C28:6; C28:7; C28:8; C28:9; C28:10; C28:11; C28:12; C28:13; C29:0; C29:1; C29:2; C29:3; C29:4; C29:5; C29:6; C29:7; C29:8; C29:9; C29:10; C29:11; C29:12; C29:13; C30:0; C30:1; C30:2; C30:3; C30:4; C30:5; C30:6; C30:7; C30:8; C30:9; C30:10; C30:11; C30:12; C30:13; C30:14; C31:0; C31:1; C31:2; C31:3; C31:4; C31:5; C31:6; C31:7; C31:8; C31:9; C31:10; C31:11; C31:12; C31:13; C31:14; C32:0; C32:1; C32:2; C32:3; C32:4; C32:5; C32:6; C32:7; C32:8; C32:9; C32:10; C32:11; C32:12; C32:13; C32:14; C32:15; C33:0; C33:1; C33:2; C33:3; C33:4; C33:5; C33:6; C33:7; C33:8; C33:9; C33:10; C33:11; C33:12; C33:13; C33:14; C33:15; C34:0; C34:1; C34:2; C34:3; C34:4; C34:5; C34:6; C34:7; C34:8; C34:9; C34:10; C34:11; C34:12; C34:13; C34:14; C34:15; C34:16; C35:0; C35:1; C35:2; C35:3; C35:4; C35:5; C35:6; C35:7; C35:8; C35:9; C35:10; C35:11; C35:12; C35:13; C35:14; C35:15; C35:16; C36:0; C36:1; C36:2; C36:3; C36:4; C36:5; C36:6; C36:7; C36:8; C36:9; C36:10; C36:11; C36:12; C36:13; C36:14; C36:15; C36:16; C36:17; C37:0; C37:1; C37:2; C37:3; C37:4; C37:5; C37:6; C37:7; C37:8; C37:9; C37:10; C37:11; C37:12; C37:13; C37:14; C37:15; C37:16; C37:17; C38:0; C38:1; C38:2; C38:3; C38:4; C38:5; C38:6; C38:7; C38:8; C38:9; C38:10; C38:11; C38:12; C38:13; C38:14; C38:15; C38:16; C38:17; C38:18; C39:0; C39:1; C39:2; C39:3; C39:4; C39:5; C39:6; C39:7; C39:8; C39:9; C39:10; C39:11; C39:12; C39:13; C39:14; C39:15; C39:16; C39:17; C39:18; C40:0; C40:1; C40:2; C40:3; C40:4; C40:5; C40:6; C40:7; C40:8; C40:9; C40:10; C40:11; C40:12; C40:13; C40:14; C40:15; C40:16; C40:17; C40:18; or C40:19.

13-18. (canceled)

19. The double-stranded RNA molecule of claim 5, wherein the linker is a dinucleotide.

20-22. (canceled)

23. The double-stranded RNA molecule of claim 1, wherein the first strand consists of the contiguous nucleotide sequence.

24-30. (canceled)

31. The double-stranded RNA molecule of claim 2, wherein the AHA-1 target sequence consists of SEQ ID NO: 1 or SEQ ID NO: 2.

32-36. (canceled)

37. The double-stranded RNA molecule of claim 2. [36,] wherein the one or more modified nucleoside is a 2′ sugar modified nucleoside(s) independently selected from the group consisting of: 2′-O-alkyl-RNA; 2′-O-methyl RNA (2′-OMe); 2′-O-methoxyethyl-RNA (2′-MOE), 2′-alkoxy-RNA; 2′-amino-DNA; 2′-fluro-RNA; 2′-fluoro-DNA; arabino nucleic acid (ANA); 2′-fluoro-ANA; locked nucleic acid (LNA), and any combination thereof.

38. The double-stranded RNA molecule of claim 37, wherein the 2′ sugar modified nucleoside is an affinity enhancing 2′ sugar modified nucleoside.

39. The double-stranded RNA molecule of claim 1, wherein one or more of the internucleoside linkages positioned between the nucleosides on the contiguous nucleotide sequence are modified.

40. The double-stranded RNA molecule of claim 39, wherein:
(i) at least about 75%, at least about 80%, at least about 85%, at least about 90%, at least about 95%, or about 100% of the internucleoside linkages positioned between the nucleosides on the contiguous nucleotide sequence are modified;
(ii) one or more, or all, of the modified internucleoside linkages comprise a phosphorothioate linkage; and/or
(iii) all the internucleoside linkages present in the double-stranded RNA molecule are phosphorothioate internucleoside linkages.

41-44. (canceled)

45. A pharmaceutical composition comprising the double-stranded RNA molecule of claim 1 and a pharmaceutically acceptable diluent, solvent, carrier, salt, and/or adjuvant.

46. A method for treating or preventing a disease in a subject comprising administering a therapeutically or prophylactically effective amount of the double-stranded RNA molecule of claim 1 to a subject in need thereof.

47-48. (canceled)

49. The method of claim 46, wherein:
(i) the disease is conjunctivitis, dry eyes or inflammation;
(ii) the double-stranded RNA molecule is administered to one eye or to both eyes once per day, twice per day, three times per day, or more than three times per day; and/or
(iii) the double-stranded RNA molecule is administered to one eye or to both eyes for:
(a) less than one day, one day, two days, three days, four days, five days, six days, seven days, or more;
(b) the double-stranded RNA molecule is administered to one eye or to both eyes for one week, two weeks, three weeks, four weeks, five weeks, six weeks or more than six weeks, or (ii) one month. two months, three months, four months, five months, six months, or more than 6 months; or
(c) the double-stranded RNA molecule is administered to one eye or to both eyes for one year, two years, three years, four years, five years or more than five years.

50-52. (canceled)

53. An in vitro method for modulating expression of a target in a cell, the method comprising administering the double-stranded RNA molecule of claim 1 to the cell in an effective amount.

54. The double-stranded RNA molecule of claim 19, wherein the dinucleotide is CA.
